(19) United States
(12) Patent Application Publication
(10) Pub. No.: US 2024/0209398 A1
(43) Pub. Date: Jun. 27, 2024

(54) FLOW CHANNEL STRUCTURE AND METHOD FOR PRODUCING LIPID PARTICLE

(71) Applicant: KABUSHIKI KAISHA TOSHIBA, Tokyo (JP)

(72) Inventors: Shu OKUMURA, Oyama (JP); Masato AKITA, Kawasaki (JP); Mitsuko ISHIHARA, Setagaya (JP); Mitsuaki KATO, Kawasaki (JP)

(73) Assignee: KABUSHIKI KAISHA TOSHIBA, Tokyo (JP)

(21) Appl. No.: 18/591,102

(22) Filed: Feb. 29, 2024

Related U.S. Application Data
(63) Continuation of application No. PCT/JP2023/007850, filed on Mar. 2, 2023.

(30) Foreign Application Priority Data
Sep. 15, 2022 (JP) 2022-147321

(51) Int. Cl. C12N 15/88; B01L 3/00

(52) U.S. Cl. CPC C12N 15/88; B01L 3/502761; B01L 2200/0647; B01L 2200/16; B01L 2300/0816; B01L 2300/087; B01L 2300/0877

(57) ABSTRACT

According to one embodiment, a flow channel structure includes a first flow channel, a second flow channel, a third flow channel, a first merging portion that connects one end of the first flow channel, one end of the second flow channel, and one end of the third flow channel to one another, a fourth flow channel, a fifth flow channel, and a second merging portion that connects the other end of the third flow channel, one side end of the fourth flow channel, and one end of the fifth flow channel to one another. The one end of the first flow channel of the flow channel structure has a first shallow portion shallower than the first merging portion, and the one end of the fourth flow channel has a second shallow portion shallower than the second merging portion.

### CROSS-REFERENCE TO RELATED APPLICATIONS

This application is a Continuation Application of PCT Application No. PCT/JP2023/007850, filed Mar. 2, 2023 and based upon and claims the benefit of priority from Japanese Patent Application No. 2022-147321, filed Sep. 15, 2022, the entire contents of all of which are incorporated herein by reference.

### REFERENCE TO A SEQUENCE LISTING

In accordance with 37 CFR § 1.831, the present specification makes reference to a Sequence Listing submitted electronically as a .xml file named “552181US_ST26. xml”. The .xml file was generated on Feb. 27, 2024 and is 7,463 bytes in size. The entire contents of the Sequence Listing are hereby incorporated by reference.

### FIELD

Embodiments described herein relate generally to a flow channel structure and a method for producing a lipid particle.

### BACKGROUND

Conventionally, the production of lipid particles is performed by two steps; a mixing step of mixing a lipid constituting lipid particles and a substance serving as an encapsulated product of the lipid particles in an organic solvent and a diluting step of diluting a solution of the organic solvent obtained in the mixing step with an aqueous solution to form lipid particles.

However, the particle size of the lipid particles obtained according to the conventional method may not be uniform. When the particle size of the lipid particles is non-uniform, a problem arises in that the stability of the effect as a preparation containing the lipid particles is insufficient.

### BRIEF DESCRIPTION OF THE DRAWINGS

FIG. 1 is a plan view and a cross-sectional view illustrating an example of a flow channel structure of a first embodiment.

FIG. 2 is a perspective view illustrating an example of the flow channel structure of the first embodiment.

FIG. 3 is a cross-sectional view illustrating an example of a flow channel cross-section of the flow channel structure of the first embodiment, in which part (a) of FIG. 3 illustrates a cross-sectional shape of a first swirl structure, and part (b) of FIG. 3 illustrates a cross-sectional shape of a second swirl structure.

FIG. 4 is a cross-sectional view illustrating an example of the flow channel cross-section of the flow channel structure of the first embodiment, in which part (a) of FIG. 4 illustrates a square flow channel cross-section, part (b) of FIG. 4 illustrates a substantially square flow channel cross-section in which two corners of a bottom portion are rounded, and part (c) of FIG. 4 illustrates a rounded flow channel cross-section of which radius is a distance of a cross-section of half a side of a square.

FIG. 5 is a plan view illustrating an example of a flow channel structure of a second embodiment.

FIG. 6 is a plan view illustrating an example of a flow channel structure of a third embodiment.

FIG. 7 is a plan view illustrating an example of a flow channel structure of a fourth embodiment, in which part (a) of FIG. 7 illustrates a flow channel structure including a plurality of mixing units connected in series, part (b) of and FIG. 7 illustrates a flow channel structure in which a first diverging and merging flow channel and a second diverging and merging flow channel of some of the mixing units are arranged in an inverted manner.

FIG. 8 is a plan view illustrating an example of the flow channel structure of the fourth embodiment, in which part (a) of FIG. 8 illustrates a flow channel structure in which a plurality of mixing units are connected in parallel, and part (b) of FIG. 8 illustrates a flow channel structure in which a plurality of mixing units are connected in series and in parallel.

FIG. 9 is a plan view illustrating an example of the flow channel structure of the fourth embodiment.

FIG. 10 is a cross-sectional view illustrating an example of a flow channel structure of a fifth embodiment, in which part (a) of FIG. 10 illustrates a flow channel structure including a flow channel longer than other flow channels, and part (b) of FIG. 10 illustrates a flow channel structure including a flow channel wider than other flow channels.

FIG. 11 is a plan view illustrating an example of a flow channel structure of a seventh embodiment.

FIG. 12 is a cross-sectional view illustrating an example of a flow channel cross-section in a trap structure of the flow channel structure of the seventh embodiment.

FIG. 13 is a perspective view illustrating a state of the trap structure when a fluid containing a foreign substance is caused to flow in the trap structure of the flow channel structure of the seventh embodiment.

Part (a) and (b) of FIG. 14 are plan views each illustrating an example of the trap structure of the flow channel structure of the seventh embodiment.

FIG. 15 is a view according to a method for producing a flow channel structure of an embodiment, in which part (a) to (e) of FIG. 15 are cross-sectional views each illustrating an example of a flow channel structure produced by the method.

FIG. 16 is an image showing a lipid particle of an embodiment.

FIG. 17 is a flowchart illustrating a method for producing a lipid particle of an embodiment.

FIG. 18 is a plan view illustrating an example of a flow channel structure used in the method for producing a lipid particle of the embodiment, in which part (a) of FIG. 18 illustrates an condensation flow channel of the flow channel structure, part (b) of FIG. 18 illustrates a mixing flow channel of the flow channel structure, part (c) of FIG. 18 illustrates a granulation flow channel of the flow channel structure, and part (d) of FIG. 18 illustrates a concentration flow channel of the flow channel structure.

FIG. 19 is a schematic plan view of a flow channel structure used in Experiment of Example 1, in which part (a) of FIG. 19 illustrates a flow channel structure of Example, and part (b) of FIG. 19 illustrates a flow channel structure of Comparative Example 1 and 2.

Part (a) of FIG. 20 is a schematic view of a flow channel structure of Comparative Example 3 used in Experiment of Example 2, and part (b) of FIG. 20 illustrates a laminar flow structure of the flow channel structure of Comparative Example 3.

FIG. 21 is a schematic view of a second swirl structure of a flow channel structure used in Experiment Example 3, in which part (a) of FIG. 21 is a schematic plan view illustrating a flow channel structure in which a fourth flow channel includes a shallow portion, part (b) of FIG. 21 is a schematic plan view illustrating a flow channel structure in which not only a part of the fourth flow channel but also the entire region of the fourth flow channel is shallower than a depth of a fifth flow channel, and part (c) of FIG. 21 is a flow channel structure in which the entire fourth flow channel is shallower than the depth of the fifth flow channel and is wider than a width of the fifth flow channel.

### DETAILED DESCRIPTION

In general, according to one embodiment, a flow channel structure includes: a first flow channel; a second flow channel; a third flow channel; a first merging portion that connects one end of the first flow channel, one end of the second flow channel, and one end of the third flow channel to one another; a fourth flow channel; a fifth flow channel; and a second merging portion that connects the other end of the third flow channel, one side end of the fourth flow channel, and one end of the fifth flow channel to one another. The one end of the first flow channel of the flow channel structure according to the embodiment has a first shallow portion that is shallower than a depth of the first merging portion, and the one end of the fourth flow channel has a second shallow portion that is shallower than a depth of the second merging portion.

Embodiments will be described hereinafter with reference to the accompanying drawings. Note that in the specification and drawings, the same elements as those described in connection with preceding drawings are denoted by like reference numerals, and a detailed description thereof is omitted unless otherwise necessary. In addition, in some cases, in order to make the description clearer, the widths, thicknesses, shapes, etc., of the respective parts are schematically illustrated in the drawings, compared to the actual modes.

### First Embodiment

A flow channel structure according to a first embodiment includes: a first flow channel; a second flow channel; a third flow channel; a first merging portion that connects one end of the first flow channel, one end of the second flow channel, and one end of the third flow channel to one another; a first shallow portion that is located at the one end of the first flow channel and is shallower than a depth of the first merging portion; a fourth flow channel; a fifth flow channel; a second merging portion that connects the other end of the third flow channel, one end of the fourth flow channel, and one end of the fifth flow channel to one another; and a second shallow portion that is located at the one end of the fourth flow channel and is shallower than a depth of the second merging portion.

As will be described below, the first merging portion where the first flow channel, the second flow channel, and the third flow channel are connected to one another and the second merging portion where the third flow channel, the fourth flow channel, and the fifth flow channel are connected to one another have a structure that generates a swirling flow (swirling). That is, the flow channel structure according to the first embodiment is a flow channel structure in which two swirl structures are connected. Hereinafter, of the two swirl structures of the flow channel structure of the first embodiment, the upstream side swirl structure is referred to as a “first swirl structure”, and the downstream side swirl structure is referred to as a “second swirl structure”. The upstream side refers to a side to which a fluid is delivered when the flow channel structure of the embodiment is used, and conversely, the downstream side refers to a side to which the fluid is delivered.

Therefore, when description is made based on a flow direction of the fluid, the flow channel structure according to the first embodiment is a flow channel structure having a first flow channel, a second flow channel, a third flow channel, a fourth flow channel, and a fifth flow channel. A downstream end of the first flow channel, a downstream end of the second flow channel, and an upstream end of the third flow channel are connected to one another in the first merging portion, and a downstream end of the third flow channel, a downstream end of the fourth flow channel, and an upstream end of the fifth flow channel are connected to one another in the second merging portion. The downstream end of the first flow channel has a first region that is shallower than a depth of the first merging portion, and the downstream end of the fourth flow channel has a second region that is shallower than a depth of the second merging portion.

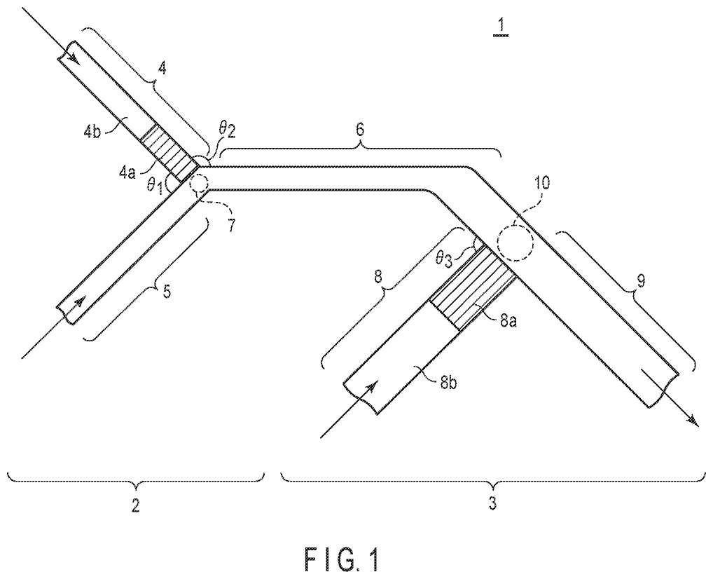

For example, as illustrated in FIG. 1, the flow channel structure 1 of the first embodiment includes a first swirl structure 2 and a second swirl structure 3, and the first swirl structure 2 and the second swirl structure 3 are connected by a flow channel that does not directly communicate with an external space of the flow channel structure 1. Hereinafter, the first swirl structure 2 and the second swirl structure 3 will be described in detail.

In the present specification, the “flow channel” is defined as a space formed inside the flow channel structure that has openings at an upstream end and a downstream end, respectively, and the space is defined such that a liquid-tight base material constituting the flow channel structure is a wall surface and a top surface thereof is sealed by the base material of the flow channel structure. The expression that two flow channels are “connected” to each other refers to a state where an opening at a downstream end (or an upstream end) of one flow channel and an opening at an upstream end (or a downstream end) of the other flow channel are liquid-tightly connected so as to communicate with internal spaces of the both flow channels, and a continuous space is formed.

The first swirl structure 2 includes the first flow channel 4, the second flow channel 5, and the third flow channel 6, and is a flow channel system formed by connecting a downstream end of the first flow channel 4 and a downstream end of the second flow channel 5 to an upstream end of the third flow channel 6. Upstream ends of the first flow channel 4 and the second flow channel 5 each have an opening (not illustrated), and can communicate with the external space of the flow channel structure 1 via each opening. When the external space communicating with each opening is sealed from air, internal spaces of the first flow channel 4 and the second flow channel 5 are also sealed systems (sealed from air). For example, a tank or a pump (not illustrated) that stores a fluid can be connected to such openings of the first flow channel 4 and the second flow channel 5 to supply a fluid. The third flow channel 6 is a flow channel whose upstream end is connected to the downstream ends of the first flow channel 4 and the second flow channel 5.

Therefore, the upstream end of the third flow channel 6 does not directly communicate with the external space of the flow channel structure 1. Hereinafter, a region of a flow channel where three of the first flow channel 4, the second flow channel 5, and the third flow channel 6 are connected and merged is referred to as a first merging portion 7.

A wall surface constituting the first merging portion 7 of the first swirl structure 2 may be either the downstream end of the second flow channel 5 and/or the upstream end of the third flow channel 6. That is, the first merging portion 7 may be located closer to the second flow channel 5 or may be located closer to the third flow channel 6, and a part of a flow channel corresponding to the first merging portion 7 may be interpreted as being included in the second flow channel 5 and the remaining part may be interpreted as being included in the third flow channel 6.

The second swirl structure 3 includes the third flow channel 6, the fourth flow channel 8, and the fifth flow channel 9, and is a flow channel system formed by connecting a downstream end of the third flow channel 6 and a downstream end of the fourth flow channel 8 to an upstream end of the fifth flow channel 9. An upstream end of the fourth flow channel 8 and a downstream end of the fifth flow channel 9 each have an opening (not illustrated), and can communicate with the external space of the flow channel structure 1 via each opening. However, when the external space communicating with each opening is a sealed system, internal spaces of the fourth flow channel 8 and the fifth flow channel 9 are also sealed systems. For example, a tank or a pump (not illustrated) that stores a fluid can be connected to such an opening of the fourth flow channel 8 to supply a fluid. On the other hand, for example, a tank (not illustrated) than can store a fluid can be connected to the opening of the fifth flow channel 9 to recover the fluid discharged from the opening. The downstream end of the third flow channel 6 is connected to the downstream end of the fourth flow channel 8 and the upstream end of the fifth flow channel 9. Therefore, the downstream end of the third flow channel 6 does not directly communicate with the external space of the flow channel structure 1. Hereinafter, a region of a flow channel where three of the third flow channel 6, the fourth flow channel 8, and the fifth flow channel 9 are connected and merged is referred to as a second merging portion 10.

A wall surface constituting the second merging portion 10 of the second swirl structure 3 may be either the downstream end of the third flow channel 6 and/or the upstream end of the fifth flow channel 9. That is, the second merging portion 10 may be interpreted as being inside the third flow channel 6 or may be interpreted as being inside the fifth flow channel 9, and a part of a flow channel corresponding to the second merging portion 10 may be interpreted as being included in the third flow channel 6 and the remaining part may be interpreted as being included in the fifth flow channel 9.

The first swirl structure 2 and the second swirl structure 3 configured as described above are connected to the third flow channel 6 configured in common to the first swirl structure 2 and the second swirl structure 3. In other words, the third flow channel 6 is a flow channel connecting the first merging portion 7 and the second merging portion 10. The third flow channel 6 is a flow channel communicating with the external space of the flow channel structure 1 via the first flow channel 4, the second flow channel 5, the fourth flow channel 8, and the fifth flow channel 9, and does not directly communicate with the external space of the flow channel structure 1. Therefore, in the sense that the third flow channel 6 does not directly communicate with the external space, the internal space thereof is isolated from the external space of the flow channel structure 1, and the system is sealed from air. The third flow channel 6 may adopt any flow channel structure as long as the internal space thereof is configured to be sealed from air. For example, the third flow channel 6 may be diverged into a plurality of flow channels between the upstream end and the downstream end thereof, and the plurality of diverged flow channels may be configured to merge into one flow channel.

In the first embodiment, the flow channel structure 1, Y shape is formed as a whole, in which the first flow channel 4 and the second flow channel 5 are connected to each other to be symmetric with respect to the long axis of the third flow channel 6 will be described as an example. As will be described below, an angle θ1 formed by the long axis of the first flow channel 4 and the long axis of the second flow channel 5 is, for example, a right angle, and at this time, an angle θ2 formed by the long axis of the first flow channel 4 and the long axis of the third flow channel 6 is, for example, 135°. Similarly, an angle θ3 formed by the fourth flow channel 8 and the fifth flow channel 9 is, for example, a right angle.

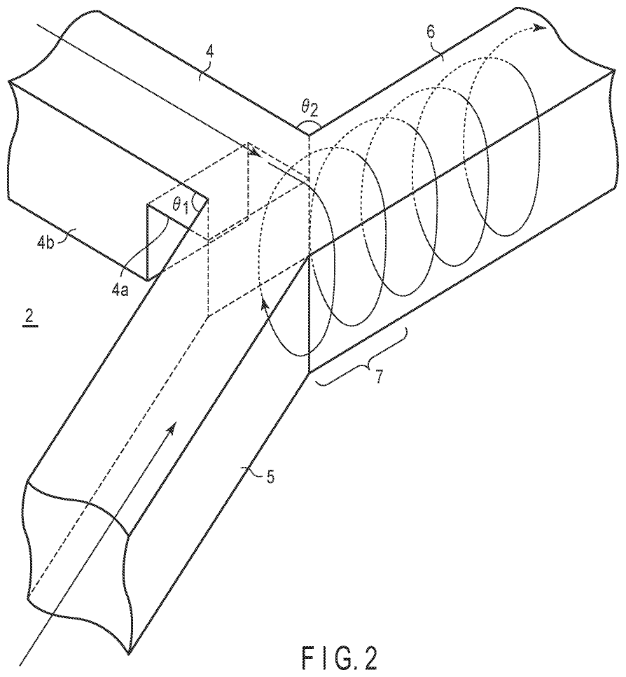

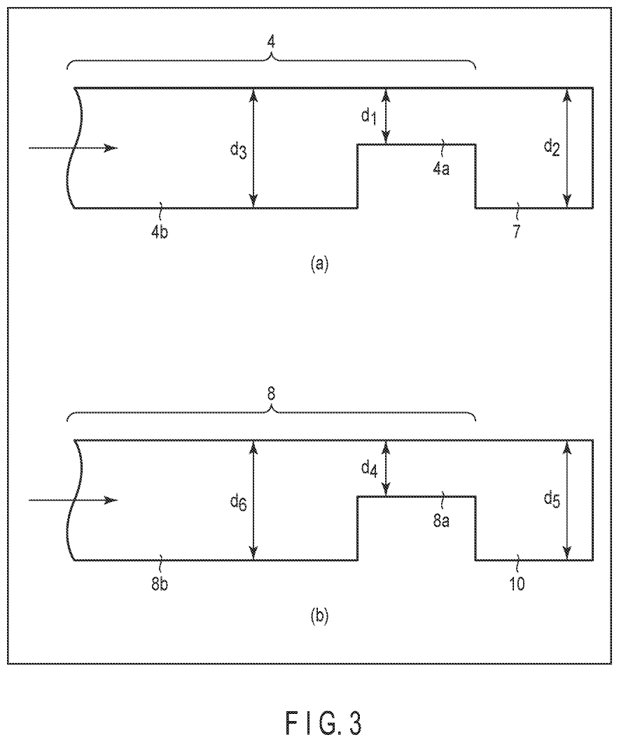

The downstream end of the first flow channel 4 has a region having a depth shallower than a flow channel in the first merging portion 7 (that is, the second flow channel 5 and/or the third flow channel 6). This region is hereinafter referred to as a “first shallow portion 4a”. FIG. 2 is a perspective view of the periphery of the first merging portion 7 of the first flow channel 4, and part (a) of FIG. 3 is a cross-sectional view from the upstream side to the downstream side. As illustrated in FIG. 2 and part (a) of FIG. 3, for example, the first shallow portion 4a has a bottom surface protruding from a region upstream of the first shallow portion 4a (hereinafter, referred to as “first deep portion 4b”) and a flow channel in the first merging portion 7, and narrows the lumen of the flow channel. The depth of the first deep portion 4b may be the same as the depth of the flow channel in the first merging portion 7.

The downstream end of the fourth flow channel 8 has a region shallower than the depth of a flow channel in the second merging portion 10 (that is, the third flow channel 6 and/or the fifth flow channel 9). This region is hereinafter referred to as a “second shallow portion 8a”. Part (a) of FIG. 3 is a cross-sectional view from the upstream to the downstream. As illustrated in part (b) of FIG. 3, for example, the second shallow portion 8a has a bottom surface protruding from a region upstream of the second shallow portion 8a (hereinafter, referred to as “second deep portion 8b”) and a flow channel in the second merging portion 10, and narrows the lumen of the flow channel. The depths of the flow channels in the second deep portion 8b and the second merging portion 10 may be the same.

In the flow channel structure 1 according to the embodiment including the flow channel configuration described above, a first fluid, and a second fluid, and a third fluid are supplied to the first flow channel 4, the second flow channel 5, and the fourth flow channel 8, respectively. At this time, a fluid is obtained by mixing the first fluid having passed through the first flow channel 4 and the second fluid having passed through the second flow channel 5, and is delivered to the third flow channel 6. A fluid is obtained by mixing the mixed fluid of the first fluid and the second fluid having passed through the third flow channel 6 and the third fluid having passed through the fourth flow channel 8, and is delivered to the fifth flow channel 9. In FIG. 1, a traveling direction of the fluid is indicated by an arrow. As illustrated in FIG. 1, the traveling direction of the fluid in the first flow channel 4 is different from that in the second flow channel 5. The traveling direction of the fluid in the fourth flow channel 8 is different from that in the fifth flow channel 9.

FIG. 2 illustrates a state when the first fluid and the second fluid are delivered to the first flow channel 4 and the second flow channel 5 of the first swirl structure 2, respectively. Arrows indicate the traveling directions of the fluids. The first fluid flowing through the first flow channel 4 passes through the first shallow portion 4a, flows into the first merging portion 7, and collides with a wall surface (that is, the second flow channel 5 or the third flow channel 6) surrounding the first merging portion 7, thereby generating a transverse vortex. The transverse vortex is a swirling flow whose rotation axis coincides with the long axis of the third flow channel 6.

As illustrated in FIG. 2, since the first fluid merges with the second fluid supplied from the second flow channel 5 at the first merging portion 7, the first fluid flows along the long axis of the third flow channel 6 while generating a transverse vortex. Due to the generation of such a transverse vortex, the first fluid and the second fluid are sufficiently mixed and stirred while flowing through the third flow channel 6. That is, since the first flow channel 4, the second flow channel 5, and the third flow channel 6 configured as described above are provided as the first swirl structure 2, the first fluid and the second fluid can be sufficiently mixed and stirred.

Since the second swirl structure 3 has the same structure as the first swirl structure 2, a swirling flow is also generated in the second swirl structure 3. In the second swirl structure 3, the second shallow portion 8a is provided in the fourth flow channel 8, and the third fluid having passed through the second shallow portion 8a collides with the fifth flow channel 9, thereby generating a transverse vortex. This transverse vortex is a swirling flow whose rotation axis coincides with the long axis of the fifth flow channel 9. Due to the generation of such a transverse vortex, the third fluid and the mixed fluid of the first fluid and the second fluid can be sufficiently mixed. That is, the mixed fluid of the first fluid, the second fluid, and the third fluid can be sufficiently stirred.

The cross-sectional area of each of the first flow channel 4, the second flow channel 5, the third flow channel 6, the fourth flow channel 8, and the fifth flow channel 9 is preferably determined according to a desired flow rate of the fluid flowing through each flow channel. For example, the cross-sectional area of each of the first flow channel 4, the second flow channel 5, the third flow channel 6, the fourth flow channel 8, and the fifth flow channel 9 may be determined to be, for example, directly proportional to the flow rate of each flow channel in order to minimize a pressure loss.

For example, since the third flow channel 6 of the flow channel structure 1 is a flow channel through which the mixed fluid of the first fluid and the second fluid is delivered, the flow rate in the third flow channel 6 is larger than the flow rate in the first flow channel 4 and the flow rate in the second flow channel 5. Therefore, the cross-sectional area of the third flow channel 6 is preferably larger than the cross-sectional area of the first flow channel 4 and the cross-sectional area of the second flow channel 5 at least immediately before the first merging portion 7. Similarly, the cross-sectional area of the fifth flow channel 9 is preferably larger than the cross-sectional area of the third flow channel 6 and the cross-sectional area of the fourth flow channel 8 at least immediately before the second merging portion 10.

As will be described below, the flow channel structure of the embodiment may be used in order to dilute the mixed fluid of the first fluid and the second fluid with the third fluid at a high magnification and to mix and stir the mixed fluid. In such a case, the flow rate of the third fluid is often higher than the flow rates of the first fluid, the second fluid, and the mixed fluid thereof. Therefore, the cross-sectional area of the fourth flow channel 8 through which the third fluid passes is preferably larger than the cross-sectional area of each of the first flow channel 4 through which the first fluid passes, the second flow channel 5 through which the second fluid passes, and the third flow channel 6 through which the mixed fluid of the first fluid and the second fluid passes.

As described above, depending on the use application of the flow channel structure 1, a difference in cross-sectional area between the flow channels merging at the second swirl structure 3 may be large. That is an asymmetric swirl structure in which cross-sectional areas are different between flow channels through which fluids are merged and connected is formed.

As illustrated in part (a) of FIG. 3, a depth d₁ of the first shallow portion 4a is preferably less than ½ of a depth d₂ of the third flow channel 6. d₁/d₂ is more preferably ⅓ or less. With such a depth, the flow velocity when the fluid flows into the third flow channel 6 from the first shallow portion 4a increases, and the transverse vortex is more likely to be generated. Similarly, as illustrated in part (b) of FIG. 3, a depth d₃ of the second shallow portion 8a is preferably less than ½ of a depth d₄ of the fifth flow channel 9. d₃/d₄ is more preferably ⅓ or less. With such a depth, the flow velocity when the fluid flows into the fifth flow channel 9 from the second shallow portion 8a increases, and the transverse vortex is more likely to be generated.

The depth d₁ of the first shallow portion 4a and the depth d₃ of the second shallow portion 8a are shallower to obtain a flow velocity that generates a larger and stronger transverse vortex, but when the depth d₁ and the depth d₃ are too shallow, an excessive pressure loss may occur, and if a foreign substance is present, blockage may occur. Therefore, the first shallow portion 4a and the second shallow portion 8a may be designed shallower by experiment or simulation (that is, d₁/d₂ or d₃/d₄ is changed to a smaller value, for example, 1/3, 1/4, 1/5 . . . ), and the shallowest depths among the depths at which the machining accuracy or pressure loss of the flow channel and the robustness against a foreign substance are appropriate may be set as d₁ and d₃.

In practice, since the accuracy of die molding or cutting, which is a preferable method used for producing the present flow channel structure, is generally 5 μm, the depth of each of the first shallow portion 4a and the second shallow portion 8a is desirably 10 μm or more at the minimum in order to avoid blockage of the flow channel due to a processing error.

In order to form a steady stable transverse vortex or in order not to cause an unexpected reaction due to cavitation, it is better to avoid reducing the depths of the first shallow portion 4a and the second shallow portion 8a to such a shallowness that the flow velocity reaches a turbulent flow range

The length of the first shallow portion 4a is preferably equal to or longer than the flow channel width of the third flow channel 6. With such a configuration, the turbulence of the flow generated when the fluid flows from the first deep portion 4b to the first shallow portion 4a can be appropriately settled in the first shallow portion 4a. By suppressing the turbulence of the flow, the transverse vortex can be more efficiently generated. Similarly, the length of the second shallow portion 8a is preferably equal to or longer than the flow channel width of the fifth flow channel 9. However, unnecessarily lengthening the first shallow portion 4a and the second shallow portion 8a may excessively increase the pressure (fluid resistance). Therefore, the length of the first shallow portion 4a is usually about three times the flow channel width at the longest. However, depending on the discharge performance of a pump, the length can also be more than three times, and such a length may be set if necessary for convenience of arrangement of the flow channel or the like.

The flow channel widths and depth of the first deep portion 4b of the first flow channel 4 and the third flow channel 6, the depth of the first shallow portion 4a, and the supply amount of the fluid are not limited, and are determined according to the type of the fluid. The fluid used for mixing and dilution is not limited to a liquid, and may be a gas. For example, for the purpose of preventing a laminar flow also in order to generate a transverse vortex, it is preferable to adjust the Reynolds number to 10 or more in a flow channel portion having a normal depth other than the first shallow portion 4a. In order to avoid turbulence for the purpose of generating a uniform transverse vortex, it is preferable to set the Reynolds number to less than 2300. More preferably, the Reynolds number is 50 to about 1000, keeping in mind the performance of commonly available pumps and the effective transverse vortex strength.

For example, when the cross-section of each of the first deep portion 4b and the third flow channel 6 is 0.3 mm square, the flow velocity is preferably about 0.5 m/s or more. Assuming that the fluid is close to water, the Reynolds number is around 100 around room temperature.

When the length of one side of the flow channel cross-section is shortened while the Reynolds number is kept constant, the pressure increases by the square. Therefore, for example, by setting the depth d₁ of the first shallow portion 4a to 0.1 mm in a flow channel of 0.3 mm on one side, the pressure loss in the first shallow portion 4a becomes around 10 times. An increase of about 10 times the pressure in the first shallow portion 4a requires limited pumps that is durable for such a condition. Therefore, the depth d₁ of the first shallow portion 4a is desirably designed to be 0.1 mm or more. In order to reduce a load on the pump, the upper limit of the increase in pressure is preferably about 10 times.

On the other hand, when a pump is used in the present flow channel structure, it is preferable to use a pump that does not cause pulsation. As such a pump, the pump having a liquid delivery amount of about 1 mL/sec can be easily obtained. In consideration of this, appropriate upper limits of the width and depth of the cross-section of each of the first deep portion 4b and the third flow channel 6 may be about 3 mm.

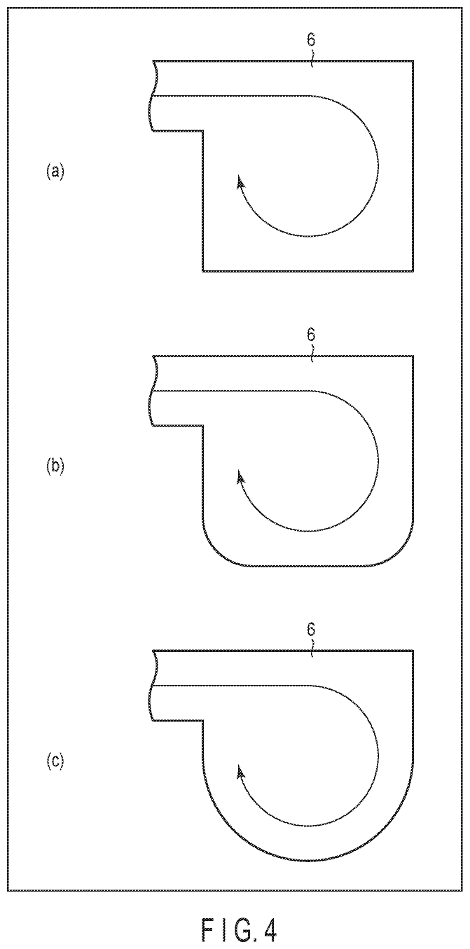

The flow channel cross-section of the third flow channel 6 is preferably a square shape having the same width and depth as illustrated in part (a) of FIG. 4. However, it is not necessary to form a square shape precisely, and a substantially square shape having one side slightly long may be used. If possible, as illustrated in part (b) of FIG. 4, a shape in which two corners of a bottom portion of the cross-section may be formed in a curved surface shape or a R-shape (i.e. rounded shape) is also preferable, or as illustrated in part (c) of FIG. 4, the bottom portion of the cross-section may be formed in a curved surface shape or a R-shape of which radius is a distance of half a side of an abbreviation square. With such a cross-sectional shape, the transverse vortex has a shape closer to a perfect circle, and the transverse vortex is maintained longer. As a result, the fluid can be well mixed and stirred. It is not necessary to have such a cross-sectional shape over the entire region of the third flow channel 6, and at least the first merging portion 7 may have such a cross-sectional shape.

As described above, since the flow channel structure of the first embodiment includes the first swirl structure and the second swirl structure that generate the transverse vortex when the fluid passes therethrough, the three types of fluids on a micro scale can be effectively mixed by supplying the three types of fluids to the first flow channel, the second flow channel, and the fourth flow channel, respectively. When different types of fluids (for example, fluids in which respective solutes and/or solvents are different from each other) are mixed and stirred, the respective fluids will be diluted compared to before mixing. At this time, the fluids can be more uniformly diluted by effectively mixing and stirring the fluids. When the fluid is supplied to the flow channel structure of the embodiment, a sealed system tank or pump is connected to the plurality of flow channels of the flow channel structure, so that the internal space of each of the plurality of flow channels of the flow channel structure is a sealed system. As described above, the plurality of flow channels are also liquid-tightly connected to one another. That is, it is possible to dilute the fluid in the sealed system by using the flow channel structure of the embodiment.

Conventionally, the production of lipid particles is performed by mixing a lipid constituting lipid particles and a substance serving as an encapsulated product of the lipid particles in an organic solvent, and further, diluting a solution of the organic solvent obtained by mixing with an aqueous solution. The dilution operation is generally performed in an open system, but the particle size of lipid particles obtained through the dilution operation in such a open system tends to be uneven, which has been a problem in production of a product containing lipid particles whose particle size uniformity is important for the quality of the product.

Recently, the inventors have found that the amount of water vapor in the working environment is one of factors for determining the particle size of lipid particles and that production of lipid particles in the working environment of the open system, in which the amount of water vapor fluctuates, is one of causes of the above problems.

Since the dilution can be completed in the sealed system by using the flow channel structure of the embodiment, it is possible to eliminate the influence of the amount of water vapor in the working environment, and thus, it is possible to produce lipid particles having a uniform particle size with excellent efficiency.

### Second Embodiment

A flow channel structure according to a second embodiment is different from the flow channel structure of the first embodiment in that the flow channel structure further includes a swirl structure. That is, the flow channel structure of the second embodiment is a flow channel structure including at least three or more swirl structures. Hereinafter, description of configurations similar to those of the flow channel structure described in the first embodiment will be omitted.

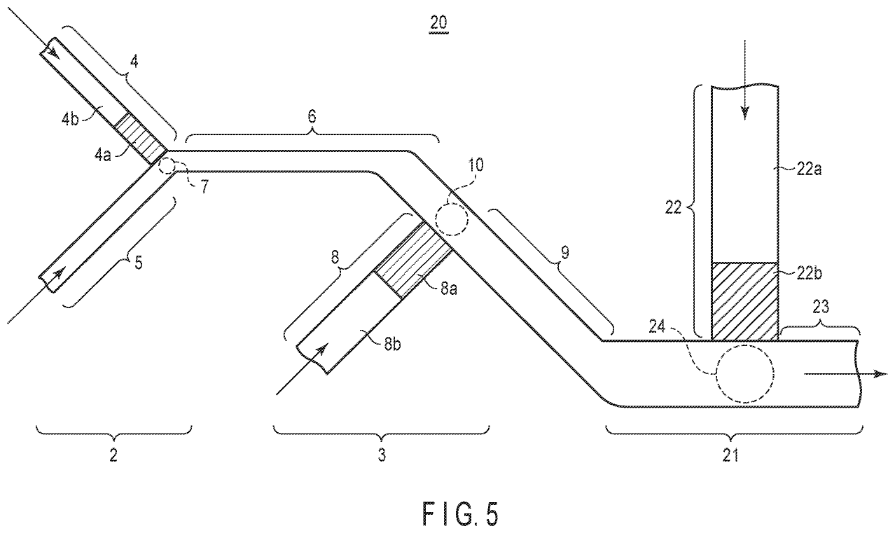

For example, in a flow channel structure 20 illustrated in FIG. 5, a third swirl structure 21 is connected to a downstream side of a second swirl structure 3, that is, a fifth flow channel 9. The third swirl structure 21 includes the fifth flow channel 9, a sixth flow channel 22, and a seventh flow channel 23, and is a flow channel system including a liquid-tight and sealed space formed by connecting a downstream end of the fifth flow channel 9 and a downstream end of the sixth flow channel 22 to an upstream end of the seventh flow channel 23.

An upstream end of the sixth flow channel 22 and a downstream end of the seventh flow channel 23 each have an opening (not illustrated), and can communicate with the external space of the flow channel structure 1 via each opening. However, when the external space communicating with each opening is a sealed system, internal spaces of the sixth flow channel 22 and the seventh flow channel 23 are also sealed systems. For example, a tank or a pump (not illustrated) that stores a fluid can be connected to such an opening of the sixth flow channel 22 to supply a fluid. On the other hand, for example, a tank (not illustrated) that can store a fluid can be connected to the opening of the seventh flow channel 23 to recover the fluid discharged from the opening. The downstream end of the fifth flow channel 9 is connected to the downstream end of the sixth flow channel 22 and the upstream end of the seventh flow channel 23. Therefore, the downstream end of the fifth flow channel 9 does not directly communicate with the external space of the flow channel structure 1.

Hereinafter, a region of a flow channel where the fifth flow channel 9, the sixth flow channel 22, and the seventh flow channel 23 are connected and merged is referred to as a “third merging portion 24”. A wall surface constituting the third merging portion 24 may be either the downstream end of the fifth flow channel 9 and/or the upstream end of the seventh flow channel 23. That is, the third merging portion 24 may be interpreted as being inside the fifth flow channel 9 or may be interpreted as being inside the seventh flow channel 23, and a part of a flow channel corresponding to the third merging portion 24 may be interpreted as being included in the fifth flow channel 9 and the remaining part may be interpreted as being included in the seventh flow channel 23.

A region shallower than the depth of a flow channel corresponding to the third merging portion 24 (that is, the fifth flow channel 9 and/or the seventh flow channel 23) is formed at the downstream end of the sixth flow channel 22. This region is hereinafter referred to as a “third shallow portion 22a”. The third shallow portion 22a has a bottom surface protruding from a region upstream of the third shallow portion 22a of the sixth flow channel 22 (hereinafter, referred to as “third deep portion 22b”) and a flow channel corresponding to the third merging portion 24, and narrows the lumen of the flow channel. For example, the depths of flow channels corresponding to the third deep portion 22b and the third merging portion 24 may be the same.

When a fluid that is different from each fluid passing through the first flow channel 4, the second flow channel 5, and the fourth flow channel 8 is delivered to the sixth flow channel 22 of the flow channel structure 20 having the above configuration, the four types of fluids can be sufficiently mixed, stirred, and diluted in the sealed system by the transverse vortex generated by the third swirl structure 21.

As a further embodiment, by further including at least one or more flow channels having a shallow portion on the downstream side of the sixth flow channel 22 and the seventh flow channel 23 and further including at least one or more flow channels to which the upstream end is connected at the downstream end of the flow channel having a shallow portion, the flow channel structure 20 may further include at least one or more additional swirl structure.

For example, assuming that the flow channel structure of the second embodiment has, for example, the first flow channel 4, the fourth flow channel 8, the sixth flow channel 22, . . . , and the X-th flow channel (here, X is an even number of 8 or more, and is a total of (X/2)) as the flow channel having a shallow portion, a total of (X/2) swirl structure groups (for example, the first swirl structure 2, the second swirl structure 3, the third swirl structure 21, . . . , the (X/2)-th swirl structure) may be formed by including a total of (X/2+1) flow channels (for example, the second flow channel 5, the third flow channel 6, the fifth flow channel 9, . . . , the (X+1)-th flow channel) merging with the flow channel having a shallow portion.

According to such a flow channel structure, it is possible to mix, stir, and dilute different types of fluids (for example, the first fluid, the second fluid, the third fluid, . . . , the (X/2+1)-th fluid) in the sealed system.

When description is made based on the flow channel structure 1 of the first embodiment, the flow channel structure further includes flow channels A (total number is m, hereinafter, m is any integer of 1 or more) having a shallow portion on another end side of the fifth flow channel and flow channels B (total number is m) each of which is connected to each of the flow channels A. When each of the flow channels A (total number is m) is represented as a (2n+4)-th flow channel (hereinafter, n is an integer of 1 to m), and each of the flow channels B (total number is m) is represented as a (2n+5)-th flow channel, the other end of a (2n+3)-th flow channel, one end of the (2n+4)-th flow channel, and one end of the (2n+5)-th flow channel are connected to one another in a (n+2)-th merging portion. The one end of the (2n+4)-th flow channel has a (n+2)-th shallow portion that is shallower than a depth of the (n+2)-th merging portion.

When description is made based on a flow direction of the fluid, the flow channel structure of the second embodiment including at least one or more additional swirl structures further includes the flow channels A (total number is m) having a shallow portion in addition to the first flow channel 4 and the fourth flow channel 8, and further includes the flow channels B (total number is m) having an upstream end connected to a downstream end of the flow channels A in addition to the second flow channel 5, the third flow channel 6, and the fifth flow channel 9. Here, m is an integer of 1 or more.

When each of the flow channels A (total number is m) is represented as a (2n+4)-th flow channel (hereinafter, n is an integer of 1 to m), each of the flow channels B (total number is m) is a (2n+5)-th flow channel. At this time, the downstream end of the (2n+3)-th flow channel, the downstream end of the (2n+4)-th flow channel, and the upstream end of the (2n+5)-th flow channel of the flow channel structure are connected to one another in the (n+2)-th merging portion, and the downstream end of the (2n+4)-th flow channel has the (n+2)-th shallow portion shallower than the depth of the (n+2)-th merging portion.

For example, when m is 1, n is 1. In this case, the flow channel A is the sixth flow channel, the flow channel B is the seventh flow channel. The downstream end of the fifth flow channel, the downstream end of the sixth flow channel, and the upstream end of the seventh flow channel are connected to one another in the third merging portion, and the downstream end of the sixth flow channel has the third shallow portion shallower than the depth of the third merging portion.

For example, when m is 2, n is 1 or 2. In this case, the flow channels A are the sixth flow channel and an eighth flow channel, and the flow channels B are the seventh flow channel and a ninth flow channel. The downstream end of the fifth flow channel, the downstream end of the sixth flow channel, and the upstream end of the seventh flow channel are connected to one another in the third merging portion, and the downstream end of the sixth flow channel has the third shallow portion shallower than the depth of the third merging portion. The downstream end of the seventh flow channel, the downstream end of the eighth flow channel, and the upstream end of the ninth flow channel are connected to one another in a fourth merging portion, and the downstream end of the eighth flow channel has a fourth shallow portion shallower than the depth of the fourth merging portion.

That is, with reference to the flow channel structure 1 of the first embodiment, a flow channel structure according to a further embodiment includes additional swirl structures (total number is m). Therefore, the flow channel structure can further mix, stir, and dilute a total of m kinds of fluids in addition to the first fluid, the second fluid, and the third fluid. When such a flow channel structure is used for the production of lipid particles, a lipid solution containing a lipid of a material of the lipid particle in an organic solvent or a drug solution containing various drugs in an aqueous solvent may be supplied to each of the first flow channel, the second flow channel, the fourth flow channel, and each flow channel from a sixth flow channel to a (2m+2)-th flow channel of the flow channels A, and an aqueous solvent may be supplied to a (2m+4)-th flow channel.

### Third Embodiment

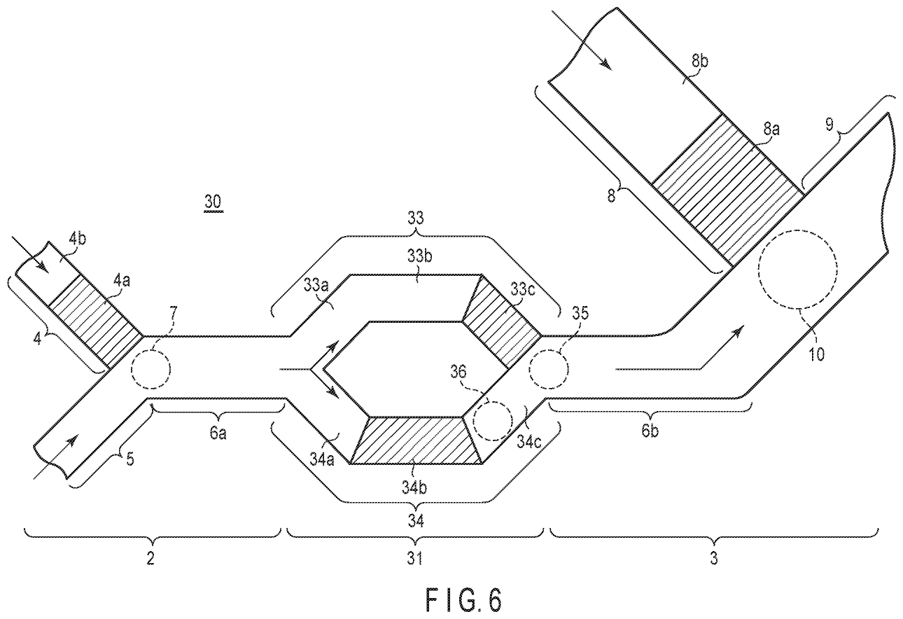

A flow channel structure according to a third embodiment is different from the flow channel structures of the first and second embodiments in that the flow channel structure further includes a mixing unit serving as a flow channel group. FIG. 6 illustrates a flow channel structure 30 that is an example of the third embodiment. In FIG. 6, for the sake of expedience, shallow portions (4a, 8a, 34b, and 33c) are indicated by a hatched line pattern. A direction in which a fluid flows is indicated by an arrow.

Specifically, in the flow channel structure according to the third embodiment, the third flow channel of the flow channel structure according to the first embodiment further includes a mixing unit. The mixing unit includes a “first diverging and merging flow channel” and a “second diverging and merging flow channel”, which are two flow channels that diverge from the third flow channel and merge into the third flow channel again, between the other end and the one end of the third flow channel. The first diverging and merging flow channel and the second diverging and merging flow channel each have a diverging portion that diverges from the third flow channel, a merging portion that merges into the third flow channel, an intermediate portion that connects the diverging portion and the merging portion and bends a connection part, a third shallow portion that is located at an end of the first diverging and merging flow channel on the merging portion side and is shallower than a depth of the third flow channel, and a fourth shallow portion that is located at the intermediate portion of the second diverging and merging flow channel and is shallower than depths of the diverging portion and the merging portion of the second diverging and merging flow channel.

When description is made based on a flow direction of the fluid, in the flow channel structure according to the third embodiment, the third flow channel of the flow channel structure according to the first embodiment further includes a mixing unit. The mixing unit includes a first diverging and merging flow channel and a second diverging and merging flow channel, which diverge from the third flow channel and merge into the third flow channel again, between the upstream end and the downstream end of the third flow channel. The first diverging and merging flow channel and the second diverging and merging flow channel each have a diverging portion that diverges from the third flow channel, a merging portion that merges into the third flow channel, an intermediate portion that connects the diverging portion and the merging portion and bends a connection part, a third shallow portion that is located at a downstream end of the merging portion of the first diverging and merging flow channel and is shallower than a depth of the third flow channel, and a fourth shallow portion that is located at the intermediate portion of the second diverging and merging flow channel and is shallower than depths of the diverging portion and the merging portion of the second diverging and merging flow channel. Details will be described below.

The flow channel structure 30 includes a first swirl structure 2 and a second swirl structure 3 similar to the flow channel structure 1 of the first embodiment, and a mixing unit 31 disposed between the first swirl structure 2 and the second swirl structure 3. In other words, with reference to the configuration of the flow channel structure 1 of FIG. 1, the mixing unit 31 is disposed to be inserted into the third flow channel 6 of the flow channel structure 1. In other words, as described in the first embodiment that “the third flow channel may adopt any flow channel structure as long as the internal space thereof is a sealed system”, the mixing unit 31 can also be interpreted as being a part of the third flow channel 6. Hereinafter, a description will be given assuming that the mixing unit 31 is disposed between the first swirl structure 2 and the second swirl structure 3 (that is, inserted into the third flow channel 6) with reference to the configuration of the flow channel structure 1 of FIG. 1.

Since the mixing unit 31 is inserted, the third flow channel 6 is divided into an upstream flow channel 6a located upstream of the mixing unit 31 and a downstream flow channel 6b located downstream of the mixing unit 31. Such an upstream flow channel 6a may be regarded as a part of the configuration of the first swirl structure 2, and the downstream flow channel 6b may be regarded as a part of the configuration of the second swirl structure 3. Hereinafter, the portions of the first swirl structure 2 and the second swirl structure 3 of the flow channel structure 30 are also collectively referred to as a “merging unit 32”.

The mixing unit 31 is a flow channel group configured to further mix and stir the fluid mixed in the first swirl structure 2 and supply the fluid to the second swirl structure 3. The mixing unit 31 includes a first diverging and merging flow channel 33 and a second diverging and merging flow channel 34. The first diverging and merging flow channel 33 and the second diverging and merging flow channel 34 each are, for example, a flow channel that diverges into two flow channels from the upstream flow channel 6a to the downstream side and merge into the downstream flow channel 6b, and the respective downstream ends are connected to the upstream end of the downstream flow channel 6b.

Hereinafter, the structures of the first diverging and merging flow channel 33 and the second diverging and merging flow channel 34 of the mixing unit 31 will be described in more detail. The first diverging and merging flow channel 33 is a flow channel including a diverging portion 33a, an intermediate portion 33b, and a merging portion 33c. The diverging portion 33a, the intermediate portion 33b, and the merging portion 33c are connected in the listed order from the upstream side to the downstream side of the flow channel structure. Similarly, the second diverging and merging flow channel 34 is a flow channel including a diverging portion 34a, an intermediate portion 34b, and a merging portion 34c, and the diverging portion 34a, the intermediate portion 34b, and the merging portion 34c are connected in the listed order from the upstream side to the downstream side of the flow channel structure.

The diverging portions 33a and 34a are both connected to the downstream end of the upstream flow channel 6a, and the fluid having passed through the first swirl structure 2 is diverted to each of the diverging portions. The intermediate portions 33b and 34b each are bent at a connection part with the downstream end of the diverging portion 33a or 34a, for example, to have an angle parallel to the long axis of the flow channel 6a. The merging portions 33c and 34c each are bent inward at a part connected to the downstream end of the intermediate portion 33b or 34b and are connected to the upstream end of the downstream flow channel 6b, and the diverted flows each having passed through the merging portions 33c and 34c merge into the downstream flow channel 6b.

The angle formed by the diverging portions 33a and 34a is not limited, and is, for example, a right angle. However, it is preferable that the angle formed by the diverging portion 33a and the upstream flow channel 6a is the same as the angle formed by the diverging portion 34a and the upstream flow channel 6a, that is, the diverging portions 33a and 34a are connected to each other symmetrically with respect to the upstream flow channel 6a as an axis. It is preferable that the diverging portions 33a and 34a have the same flow channel width and depth. When the diverging portions 33a and 34a are connected to each other symmetrically with respect to the upstream flow channel 6a as an axis and have the same flow channel width and depth, it is possible to equalize the flow rates at first diverging and merging flow channel 33 and the second diverging and merging flow channel 34. Even if the flow rates in both the diverging portions are not equal, it is possible to generate a transverse vortex in each shallow portion (in FIG. 6, the intermediate portion 34b or the merging portion 33c) downstream of the diverging portion. Therefore, the sizes or angles of the flow channels may be different from each other, but it should be considered that there is a possibility that the difficulty (required accuracy) of pressure adjustment in the flow channel having a smaller flow rate increases as the flow rate ratio increases. Thus it is preferable to diverge the fluid in approximately the same amount.

Similarly to the diverging portions 33a and 34a, it is preferable that the merging portion 33c and the merging portion 34c are connected to the downstream flow channel 6b, for example, at the same angle (that is, symmetrically to each other with respect to the downstream flow channel 6b as an axis), and have the same flow channel width and depth. The angle formed by the merging portion 33c and the merging portion 34c is, for example, a right angle.

In the first diverging and merging flow channel 33, for example, the merging portion 33c is a shallow portion having a depth of less than ½ of the depth of each of the diverging portion 33a and the intermediate portion 33b. Since the shallow portion is formed, a transverse vortex is generated near the upstream side of the downstream flow channel 6b (mixing region 35). As a result, the fluid can be further mixed and stirred. The cross-sectional shape of the flow channel of the mixing region 35 is preferably any shape illustrated in FIG. 4.

In the second diverging and merging flow channel 34, for example, the intermediate portion 34b is a shallow portion having a depth of less than ½ of the depth of the merging portion 34c. The shallow portion in the second diverging and merging flow channel 34 can be provided at the diverging portion 34a, but it is preferable to dispose the shallow portion at the intermediate portion 34b in order to further simplify the flow divergence.

Since the flow channel is bent toward the connection part between the intermediate portion 34b, which is a shallow portion, and the merging portion 34c, a transverse vortex is generated near the upstream of the merging portion 34c (mixing region 36), and the fluid can be further stirred. The cross-sectional shape of the flow channel of the mixing region 36 is preferably any shape illustrated in FIG. 4.

The mixing unit 31 may not be configured such that a shallow portion is provided in a part of the first diverging and merging flow channel 33 and the second diverging and merging flow channel 34 (for example, the merging portion 33c and the intermediate portion 34b), and instead, may be configured such that the depth of the entire region of the first diverging and merging flow channel 33 is different from the depth of the entire region of the second diverging and merging flow channel 34. The mixing unit 31 having such a configuration can mix and stir the fluid by a transverse vortex generated at the merging part between the first diverging and merging flow channel 33 and the second diverging and merging flow channel 34.

On the other hand, in the configuration in which the shallow portions are disposed in both the first diverging and merging flow channel 33 and the second diverging and merging flow channel 34 illustrated in FIG. 6, the fluid can be evenly distributed to both the diverging and merging flow channels since the pressures applied to both the diverging and merging flow channels are equal. Even if one of the diverging and merging flow channels is blocked due to a foreign substance, the fluid can be further mixed and stirred by the mixing unit 31 by letting the fluid pass through the shallow portion in the other diverging and merging flow channel to generate a transverse vortex.

When the mixing unit 31 in which the flow channel widths and depths of the first diverging and merging flow channel 33 and the second diverging and merging flow channel 34 are further narrowed is used while keeping the flow rate constant, the flow velocity of the fluid passing through the mixing unit 31 further increases. As the flow velocity increases, the speed of a transverse vortex also increase, so that the mixing and stirring effect by the mixing unit 31 can be enhanced. Therefore, when a higher mixing and stirring effect is required for the flow channel structure, it is preferable to use the mixing unit 31 in which the flow channel widths and depths of the first diverging and merging flow channel 33 and the second diverging and merging flow channel 34 are narrower. However, it should be noted that when the flow channel width and depth are too narrow, the pressure loss becomes very large, and some pumps may not function.

As described above, an example in which the mixing unit is disposed between the first swirl structure and the second swirl structure (that is, inserted into the third flow channel of the flow channel structure 1 of the first embodiment) has been described as the flow channel structure of the third embodiment, but the mixing unit is not limited to such an arrangement. As a further embodiment, the mixing unit 31 may be connected, for example, to downstream side of the second swirl structure, such as the downstream side of the fifth flow channel 9.

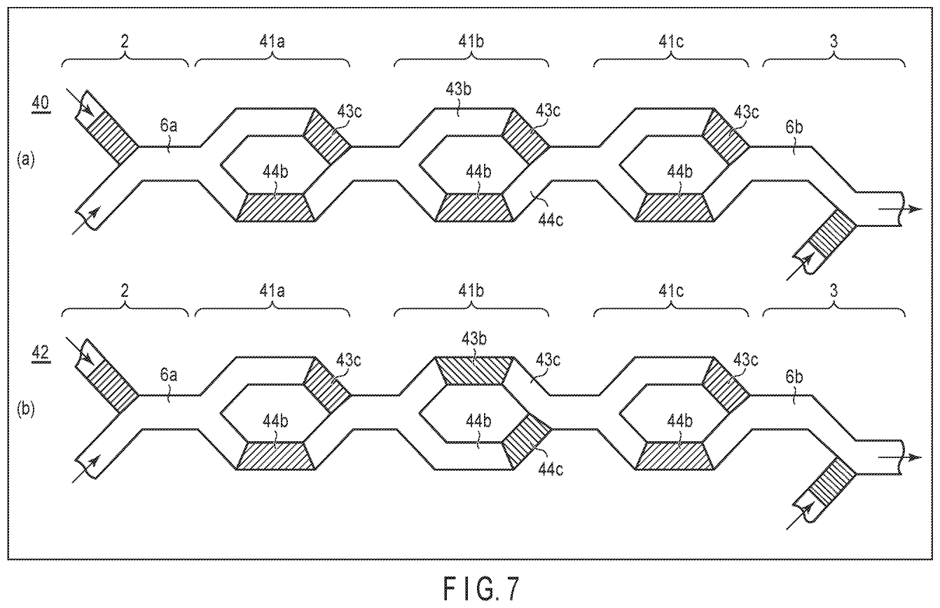

As the configuration of the mixing unit of the flow channel structure of the third embodiment, an example in which the flow channel (here, the second diverging and merging flow channel 34) disposed diagonally to the flow channel (here, the first flow channel 4) having the first shallow portion 4a of the merging unit 32 has the intermediate portion 34b as a shallow portion has been described, but the mixing unit is not limited to such a configuration. As a further embodiment, as illustrated in part (b) of FIG. 7 below, the first diverging and merging flow channel 33 and the second diverging and merging flow channel 34 may be arranged in an inverted manner.

### Fourth Embodiment

A flow channel structure according to a fourth embodiment is different from the flow channel structure of the third embodiment in that the flow channel structure includes a plurality of mixing units.

For example, as illustrated in part (a) of FIG. 7, a flow channel structure 40 includes three mixing units arranged in series, that is, a first mixing unit 41a, a second mixing unit 41b, and a third mixing unit 41c, at the downstream side of a first swirl structure 2 and the upstream side of a second swirl structure 3.

In a flow channel structure 42 illustrated in part (b) of FIG. 7, a first diverging and merging flow channel 43 (including a shallow portion at a merging portion 43c) and a second diverging and merging flow channel 44 (including a shallow portion at an intermediate portion 44b) are disposed so as to be inverted about an upstream flow channel 6a and a downstream flow channel 6b in the second mixing unit 41b. By alternately arranging the mixing units in which the arrangement of the first diverging and merging flow channel 43 and the second diverging and merging flow channel 44 is inverted as in this example, the fluids can be mixed more uniformly.

Although the flow channel structures illustrated in part (a) and (b) of FIG. 7 are exemplified as the flow channel structure according to the fourth embodiment, the number of the plurality of mixing units is not limited to 3, and may be 2, 4, 5, 6, or more, or the plurality of mixing units may be connected and arranged at arbitrary positions in the flow channel structure.

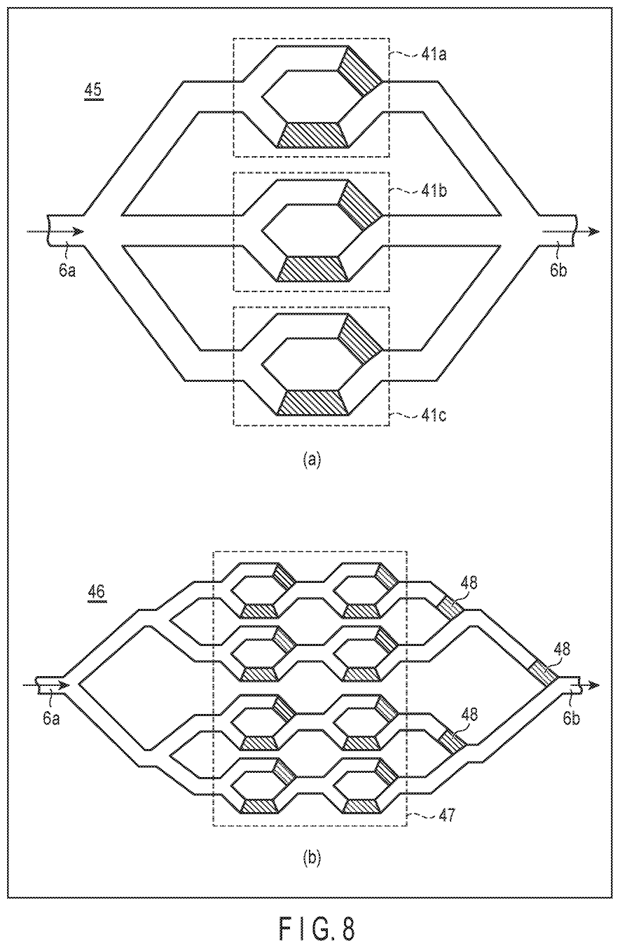

For example, a flow channel structure 45 illustrated in part (a) of FIG. 8 is also an example of the flow channel structure according to the fourth embodiment. In the flow channel structure 45, each upstream end or each downstream end of the first mixing unit 41a, the second mixing unit 42b, and the third mixing unit 42c and the upstream flow channel 6a or the downstream flow channel 6b are connected in parallel (in other words, the upstream flow channel 6a and the downstream flow channel 6b extend so as to diverge and are connected to the ends of the respective mixing units). The fluid supplied to such a flow channel structure 40 and passing through the upstream flow channel 6a is divided and delivered to each mixing unit. Each fluid passing through each mixing unit merges with the downstream flow channel 6b.

By arranging the plurality of mixing units in parallel, it is possible to reduce flow resistance to liquid delivery as compared with a case where the plurality of mixing units is arranged in series. When a pump is used for delivering liquid to the flow channel structure, it is possible to deliver a larger flow rate to the flow channel structure while reducing a load on the pump.

The flow channel structure according to the fourth embodiment may include a plurality of mixing units as a mixing unit group formed by connecting the plurality of mixing units in series and in parallel. For example, the flow channel structure 46 illustrated in part (b) of FIG. 8 includes a mixing unit group 47 arranged between the first swirl structure 2 and the second swirl structure 3. The mixing unit group 47 includes four sets of flow channel structures in which two mixing units are connected in series, and these four sets of flow channel structures are connected in parallel to the upstream flow channel 6a and the downstream flow channel 6b. In the portion where the fluids merge downstream of the mixing unit group 47, a shallow portion 48 is preferably disposed on one of the merging flow channels in order to promote mixing and stirring. The configuration of the mixing unit group 47 is not limited to the example illustrated in FIG. 8, and for example, the arrangement of the mixing units and the like can be modified according to the type or use application of the fluid. By appropriately modifying the configuration of the mixing unit group, it is possible to adjust the resistance of liquid delivery to the flow channel structure and to enhance the effect of mixing and stirring the fluid.

As described above, since the flow channel structure according to the fourth embodiment includes the plurality of mixing units, it is possible to further mix and stir the fluid as compared with a case where there is one mixing unit. By using such a flow channel structure of the fourth embodiment for the production of lipid particles, a larger amount of lipid particles can be formed with a more uniform particle size than in the case of using a flow channel structure including one mixing unit.

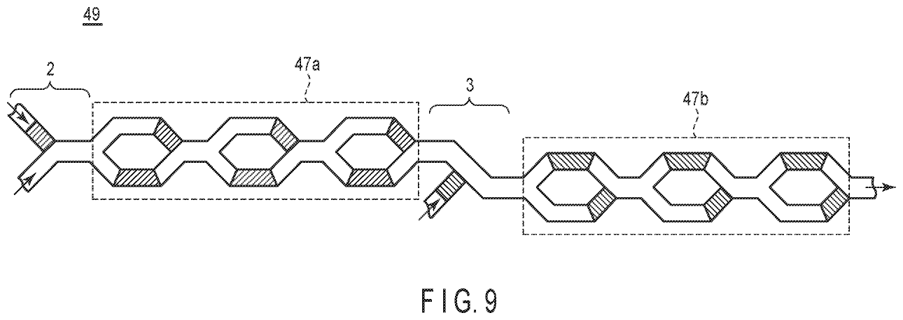

As a further embodiment, the flow channel structure may have a configuration in which the mixing unit or the mixing unit group is connected not only to the downstream end of the first swirl structure 2 but also to the downstream end of the second swirl structure 3. For example, as illustrated in FIG. 9, a flow channel structure 49 may have a configuration in which a mixing unit group 47a including, for example, three mixing units 41a, 41b, and 41c is connected to the downstream end of the first swirl structure 2 and a mixing unit group 47b including, for example, three mixing units 41d, 41e, and 41f is connected to the downstream end of the second swirl structure 3.

The flow channel structure 49 is not limited to such a configuration, and may have a configuration in which the mixing unit or the mixing unit group is connected to the downstream side of the second swirl structure 3 of the flow channel structure 42, 45, or 46 illustrated in each of part (b) of FIG. 7, and part (a) and (b) of FIG. 8.

### Fifth Embodiment

A flow channel structure according to a fifth embodiment is different from the flow channel structures of the first to fourth embodiments in that the flow channel structure further includes a flow channel which takes longer time for fluid to pass through than other flow channels constituting the flow channel structure between the first swirl structure and the second swirl structure (that is, the third flow channel further includes a flow channel which takes longer time for fluid to pass through than other flow channels).

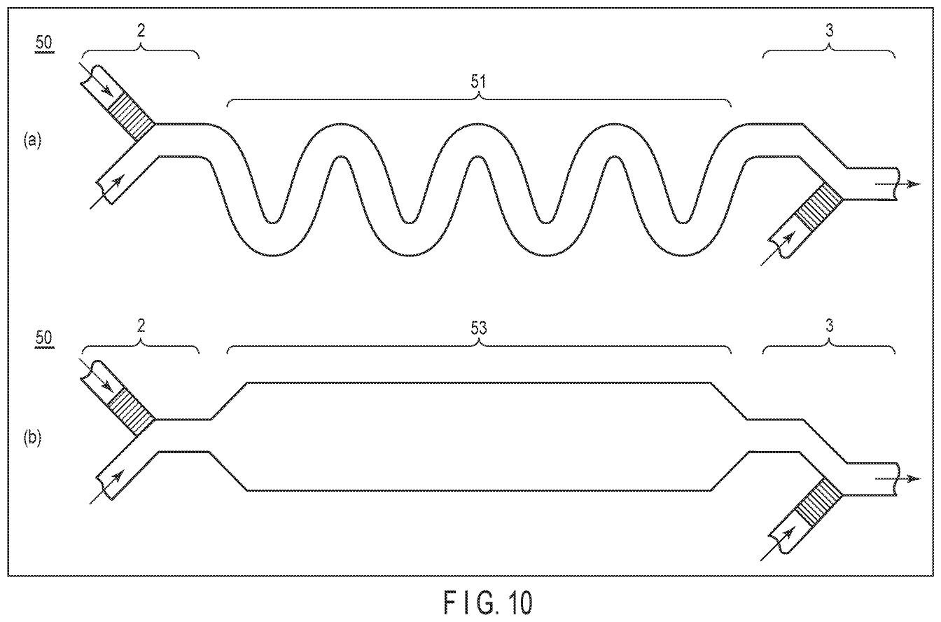

The flow channel which takes longer time for fluid to pass through described herein is, for example, a flow channel having a longer flow channel length than other flow channels, a flow channel having a wider flow channel width than other flow channels, or the like. Therefore, for example, a flow channel structure 50 according to the fifth embodiment includes, as illustrated in part (a) of FIG. 10, a third flow channel 6 includes a flow channel 51 having a longer flow channel length than other flow channels, which is connected to a downstream end of an upstream flow channel 6a and an upstream end of a downstream flow channel 6b. Alternatively, for example, a flow channel structure 52 according to the fifth embodiment includes, as illustrated in part (b) of FIG. 10, the third flow channel 6 includes a flow channel 53 having a wider flow channel width than other flow channels, which is connected to the downstream end of the upstream flow channel 6a and the upstream end of the downstream flow channel 6b.

An element (not illustrated) that applies various types of energy (for example, light, heat, sound waves, magnetism, and the like) to the flow channel 51 and the flow channel 53 is connected to the flow channel 51 and the flow channel 53 exemplified above. For example, a heater or a Peltier element is connected as an element that applies thermal energy, and for example, an ultraviolet generation element is connected as an element that applies light energy, but such an element may be disposed in the flow channel 51 or 53, or may be disposed outside the flow channel structure 50 or 52.

When an element that applies various types of energy is disposed outside the flow channel structure 50 or 52, the flow channel 51 and the flow channel 53 as a part of the third flow channel 6 are a sealed system, and thus are preferably elements capable of applying energy to the flow channel of such a sealed system. It is preferable that the flow channel is configured to have a material or structure that easily receives various types of energy according to the type of the element to be used. For example, when the ultraviolet generation element is connected as an element to the flow channel 51 or 53 that is a sealed system, irradiation with high-intensity ultraviolet rays can be performed with the passing fluid by forming the flow channel 51 or 53 using a transparent member.

Since the flow channel 51 and the flow channel 53 which takes longer time for fluid to pass through than other flow channels, various reactions can be caused to proceed with the fluid passing therethrough even while the fluid is passing through the flow channel 51 and the flow channel 53. For example, the fluid passing through the flow channel 51 and the flow channel 53 can be subjected to a physical treatment such as an ultrasonic treatment, or a chemical treatment such as a heat treatment or an ultraviolet treatment.

When the flow channel structure according to the fifth embodiment described above is used for the production of lipid particles, other steps related to the production of lipid particle (for example, a pretreatment step such as thermally denaturing a substance to be encapsulated in a lipid particle, an inspection step performed by detecting a fluorescent label, and the like) can be performed simultaneously with the production of lipid particles.

### Sixth Embodiment

A flow channel structure according to a sixth embodiment is different from the flow channel structures of the first to fifth embodiments in that the first flow channel further includes a flow channel that traps a foreign substance on the upstream side of the first shallow portion.

That is, the flow channel structure according to the sixth embodiment further includes a trap structure configured to trap a foreign substance in the first flow channel on a side closer to the other end than the first shallow portion, and a depth of the trap structure is shallower than a depth of the first flow channel on a side closer to the other end than the trap structure. When description is made based on the flow of the fluid, in the first flow channel upstream of the first shallow portion, a trap structure configured to trap a foreign substance is further provided, and the depth of the trap structure is shallower than the depth of the first flow channel on the upstream side.

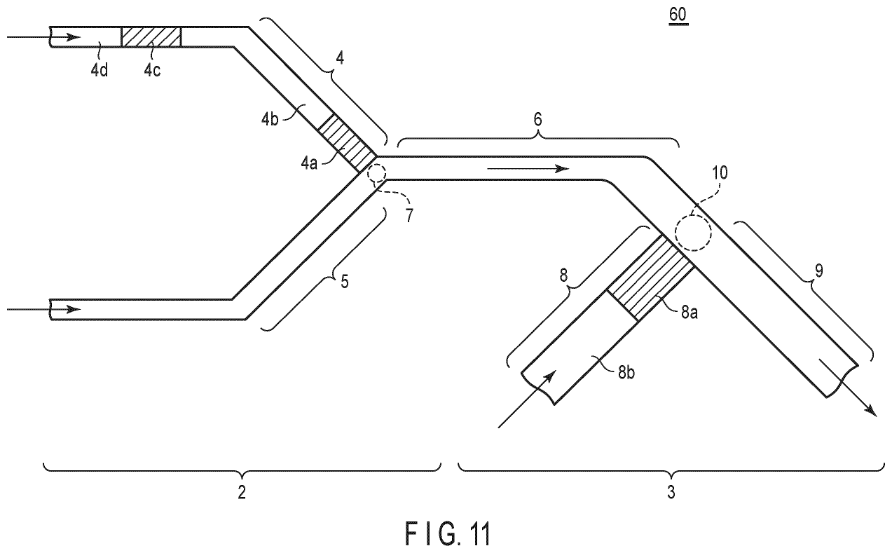

More specifically, as illustrated in FIG. 11, a first flow channel 4 of a flow channel structure 60 according to the sixth embodiment has a region shallower than a depth d₃ of a first deep portion 4b (hereinafter, referred to as “upstream shallow portion 4c”) on the upstream side of the first deep portion 4b. A depth d₇ of the upstream shallow portion 4c is shallower than a depth d₈ of a region upstream of the upstream shallow portion 4c (hereinafter, referred to as “upstream deep portion 4d”). Hereinafter, a structure including the first deep portion 4b, the upstream shallow portion 4c, and the upstream deep portion 4d is referred to as “first trap structure”.

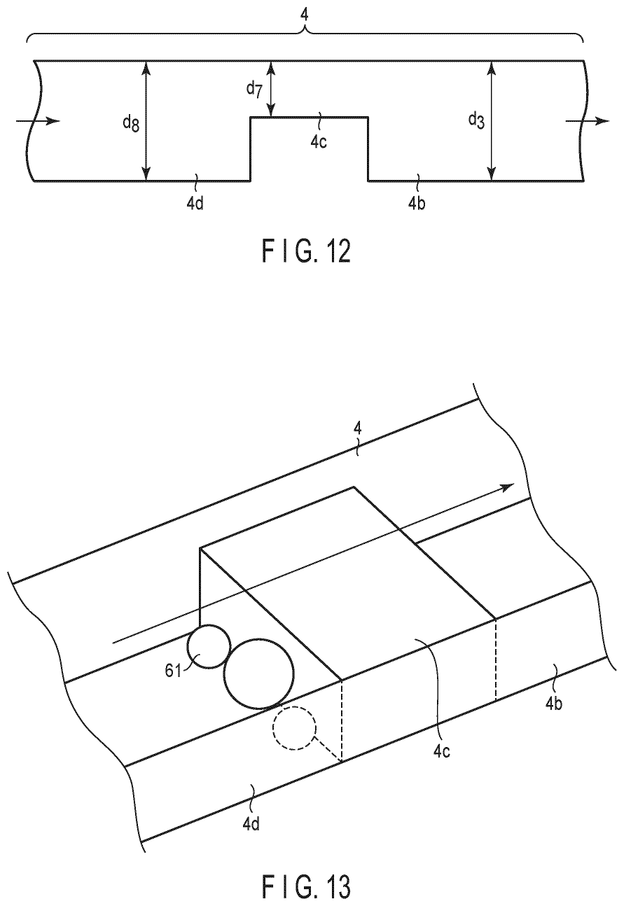

FIG. 12 is a cross-sectional view of the first flow channel 4 around the upstream shallow portion 4c. In the upstream shallow portion 4c, for example, the bottom surface protrudes toward the top surface. The depth d₈ of the upstream deep portion 4d and the depth d₃ of the first deep portion 4b may be the same as each other.

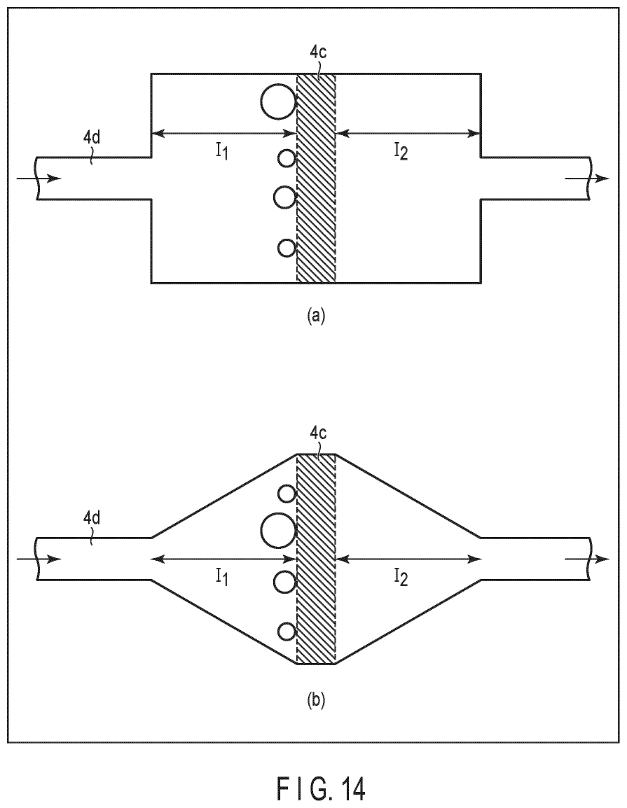

FIG. 13 illustrates a state of the first trap structure when a fluid flows. An arrow indicates the traveling directions of the fluid. When the first fluid passes through the upstream shallow portion 4c, a foreign substance 61 larger than the depth d₇ of the upstream shallow portion 4c cannot enter the upstream shallow portion 4c and is captured immediately before the upstream shallow portion 4c. As a result, the foreign substance 61 cannot enter a downstream flow structure.

The depth d₇ of the upstream shallow portion 4c is set according to an object to be removed and purpose of removal. For example, when a foreign substance having a specific size or shape adversely affects the function of another flow channel structure provided on the downstream side of the flow channel structure 60, the depth is set to a depth at which the foreign substance having the specific size or shape can be captured. When the purpose of removal is to remove a foreign substance that cannot be visually confirmed by a user of the flow channel structure 60, for example, the size of the foreign substance 61 is less than or equal to the thickness of the hair (0.1 to 0.2 mm) that is generally said to be the limit with the naked eye.

For the purpose of preventing the inflow of the foreign substance 61 having a size that stops at the first shallow portion 4a, the depth d₇ of the upstream shallow portion 4c is preferably equal to or less than that of the first shallow portion 4a. The term “same” described herein includes a case where the difference is +0.01 mm in consideration of the machining accuracy. For example, when the depth of the first flow channel 4 is 0.3 mm and the depth of the first shallow portion 4a is 0.1 mm which is ⅓ of the depth of the first flow channel 4, the depth d₇ of the upstream shallow portion 4c is preferably set to 0.1 mm or less. By providing the upstream shallow portion 4c in the first flow channel 4 in this manner, entry of the foreign substance 61 can be easily prevented, and the fluid without foreign substance 61 can flow in the flow channel structure 60.

On the other hand, even when there is a small foreign substance 61 passing through the upstream shallow portion 4c, the foreign substance 61 is unlikely to clog the first shallow portion 4a, and thus may be excluded from consideration. In this case, the depth of the shallow portion 3a may be the same as that of the shallow portion 3c. However, when it is preferable to remove such a small foreign substance 61, the depth of the upstream shallow portion 4c may be shallower than that of the first shallow portion 4a.

Since the depth d₇ of the upstream shallow portion 4c is set according to an object to be removed and purpose of removal as described above, the depth d₇ is not limited, but may be, for example, smaller than the depth d₈ of the upstream deep portion 4d. For example, the depth d₇ may be less than ½ of the depth d₈, and may be ⅓, ¼, ⅕, or the like of the depth d₈.

Since the accuracy of die molding or cutting, which is a preferable method used for producing the flow channel structure 60, is generally 5 μm, the depth d₇ of the upstream shallow portion 4c is desirably 10 μm or more at the minimum in order to avoid blockage of the flow channel due to a processing error.

The length of the upstream shallow portion 4c in a flow direction of the first fluid is preferably about the same as the flow channel widths of the first deep portion 4b and the upstream deep portion 4d. Alternatively, the length may be shorter as long as the upstream shallow portion 4c can be produced.

The flow channel width and depth and the supply amount of the fluid of each of the first deep portion 4b and the upstream deep portion 4d are not limited, and are determined according to the type of the fluid.

On the other hand, when a pump is used in the present flow channel structure, it is preferable to use a pump that does not cause pulsation from the viewpoint of producing lipid particles having a uniform particle size. As such a pump, a pump having a liquid delivery amount of about 1 ml/sec can be easily obtained. Therefore, in consideration of the availability of the pump, appropriate upper limits of the width and depth of the cross-section of each of the first deep portion 4b and the upstream deep portion 4d may be about 3 mm. For example, the depth and width of the cross-section of each of the first deep portion 4b and the upstream deep portion 4d are preferably set to 0.1 mm to 3 mm.

The flow velocity of the fluid in the first trap structure is preferably relatively small, and in order to make the flow velocity of the fluid in the first trap structure relatively slow, the cross-sectional area of the flow channel of the upstream shallow portion 4c is preferably set to be equal to or larger than that of the upstream deep portion 4d. Therefore, the width of the upstream shallow portion 4c of the flow channel structure 60 is preferably wider than those of the first deep portion 4b and the upstream deep portion 4d. The “width” described herein refers to a length of the first flow channel 4 in a direction orthogonal to the flow direction of the fluid.

For example, as illustrated in part (a) of FIG. 14, the first trap structure may maintain a wide flow channel width from the upstream shallow portion 4c to the upstream side of a length I1 and from the upstream shallow portion 4c to the downstream side of a length I2. In other words, the first flow channel 4 of the flow channel structure 60 may have a rectangular wide portion including the upstream shallow portion 4c. As illustrated in part (b) of FIG. 14, the first trap structure may be configured such that the width thereof gradually increases from the upstream deep portion 4d to the upstream side of the length I1, the width thereof becomes the widest at the upstream shallow portion 4c, and the flow channel width gradually decreases from the upstream shallow portion 4c toward the downstream side of the length I2 to the original width. In other words, the first flow channel 4 of the flow channel structure 60 may have a rhombic wide portion having the widest width at the upstream shallow portion 4c.

When the lengths I1 and I2 of the wide portions are too long, the lengths may affect the flow channel structure 60 as a dead volume, and when the lengths are too short, an increase in resistance due to the trapped foreign substance 61 may become significant. Therefore, in order to make the foreign substance 61 easily and evenly spread to the entire upstream shallow portion 4c, it is desirable that the lengths I1 and I2 of the wide portions are about the same as the width of the wide portion. The length I1 and the length I2 may be the same as or different from each other.

By setting the width of the upstream shallow portion 4c to be wide in this manner, it is possible to suppress an increase in pressure resistance in the upstream shallow portion 4c and further reduce the average flow velocity in the upstream shallow portion 4c. For example, when the depth d₇ of the upstream shallow portion 4c is 1/n (here, n is an integer of 0 or more) of the depth d₈ of the upstream deep portion 4d, by setting the flow channel width of the upstream shallow portion 4c to n times or more the flow channel width of the upstream deep portion 4d, the average flow velocity in the upstream shallow portion 4c can be set to be approximately equal to or less than the average flow velocity in the upstream deep portion 4d.

By further reducing the average flow velocity in the upstream shallow portion 4c, it is possible to reduce the possibility that the foreign substance 61 captured by the pressure increase in the upstream shallow portion 4c is pushed out and flows downstream. It is also possible to prevent an increase in pressure resistance when a large amount of the foreign substance 61 is captured by any chance. In addition, by setting the width of the upstream shallow portion 4c to be wide, the foreign substance 61 is dispersed and captured in the width direction, and it is also possible to reduce the possibility that the foreign substance 61 blocks the flow channel.

As described above, the flow channel structure of the sixth embodiment has a simple structure in which a shallow portion is provided in the first flow channel on the upstream side of the first merging portion, and can remove a foreign substance without using a complicated structure.

As a further embodiment, in the flow channel structure 60, the same structure as the first trap structure may be provided in a flow channel for introducing a fluid in which a foreign substance is expected to exist, in addition to the first flow channel. In order to prevent unexpected generation and entry of a foreign substance, it is also preferable to provide the same structure as the first trap structure in all the flow channels to be used.

For example, the trap structure may be further provided in the fourth flow channel on a side closer to the other end than the second shallow portion and/or the second flow channel on a side closer to the other end than the first merging portion. When description is made based on a flow direction of the fluid, the second flow channel 5 may include an additional trap structure upstream of the first merging portion 7 and/or the fourth flow channel 8 may include an additional trap structure upstream of the second shallow portion 8a. When the flow channel structure further includes a plurality of flow channels as described in the second embodiment (that is, when there are a plurality of flow channels having a shallow portion and a plurality of flow channels merging with the flow channels having a shallow portion), the same structure as the first trap structure may be provided in all of the plurality of flow channels or in all of the flow channels having a shallow portion among the plurality of flow channels.

As described above, by installing the plurality of trap structures on the upstream side of the flow channel of the flow channel structure, entry of a foreign substance into the flow channel can be prevented, and the performance of the flow channel can be more reliable.

### Method for Producing Flow Channel Structure

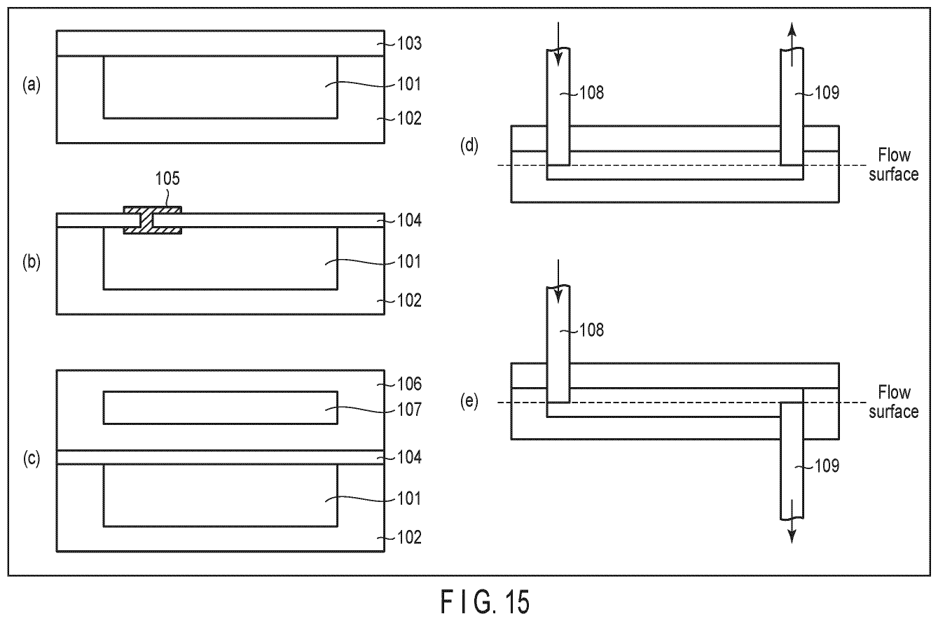

A method for producing the flow channel structure described above (hereinafter, collectively referred to as “flow channel structure 100”) will be described with reference to FIG. 15. As illustrated in part (a) of FIG. 15, the flow channel structure 100 includes, for example, a substrate 102 in which a groove 101 functioning as a flow channel is formed, and a plate-shaped lid 103 joined to the substrate 102 so as to block and seal a top surface of the groove 101.

The material for the substrate 102 may be appropriately selected from resins such as acrylic, polyethylene, and polypropylene, glass, ceramics, metal, and the like according to the use application. For example, when the flow channel structure is for medical use, a cycloolefin polymer or the like is also a preferred example. When the flow channel structure is reused many times, glass and ceramics such as quartz are preferable from the viewpoint of stability, and when a temperature and the like are adjusted, a metal having a surface subjected to a corrosion resistance treatment may be used. The groove 101 can be formed, for example, by press working or cutting using a mold. In the portion corresponding to the shallow portion, the groove 101 may be formed or cut shallower than other portions.

As the material for the lid 103, for example, the same material as that described for the substrate 102 can be used. The lid 103 may have, for example, a plate shape. Alternatively, as illustrated in part (b) of FIG. 15, a lid 104 having a thin film shape may be used.

A sensor terminal 105 configured to monitor the state of the fluid can also be attached to the lid 104 having a film shape. Alternatively, it is also possible to impart various functions or characteristics such as high thermal conductivity or a function (not illustrated) of performing a specific treatment on a specific substance to the lid 104.

When there is a concern that the lid 104 may swell due to the internal pressure, the swelling may be suppressed by pressing a pressing plate 106 from above the lid 104, as illustrated in part (c) of FIG. 15. The pressing plate 106 may include a heat medium flow channel 107 for heat exchange disposed therein, an electric terminal (not illustrated) having a sensor function, or the like.

By a simple procedure of forming the groove 101 in the substrate 102 and joining the lid 103 or 104 in this manner, the flow channel structure 100 having the internal space as a sealed system can be produced. In such a production method, for example, a step of forming grooves in both of the substrate 102 and the lid 103 and precisely aligning both, a step of forming a tunnel structure on the base material, and a step of laminating flow channels are unnecessary. Therefore, according to the production of the flow channel structure of the embodiment, it is possible to produce the flow channel structure simply and at low cost without requiring high machining accuracy at the time of production, and mass productivity is very high.

According to a further embodiment, the shallow portion may be formed by setting the depth of the portion of the groove 101 where the shallow portion is provided to the same depth as the other portions, and attaching the lid 104 having a convex portion to the corresponding portion. That is, the flow channel lumen of the shallow portion formed in this manner is recessed from above to narrow the flow channel. In such a structure, the procedure of producing such as formation and alignment of the lid is increased as compared with the above-described structure in which the bottom protrudes, but it is possible to generate the transverse vortex as described in the flow channel structure of the first embodiment.

As a further embodiment, a flow channel for introducing a fluid and a flow channel structure including a flow channel for introducing a fluid may be produced. For example, as illustrated in part (d) of FIG. 15, an introduction flow channel 108 having an end in contact with a flow channel surface having a desired depth and extending in a vertical direction, and a discharge flow channel 109 having an end in contact with the flow surface and extending in the vertical direction may be provided. When the fluid is supplied to the introduction flow channel 108 of the flow channel structure 101 illustrated in part (d) of FIG. 15, the fluid passes through the introduction flow channel 108 in the direction of gravity, passes through the groove portion 101, then passes through the discharge flow channel 109 in the direction opposite to the direction of gravity, and is discharged.

As illustrated in part (e) of FIG. 15, the discharge flow channel 109 may be provided so as to extend in the direction of gravity from the flow surface. In this case, the fluid having passed through the groove portion 101 passes through the inside of the discharge flow channel 109 in the direction of gravity and is discharged. By utilizing the height difference provided by the discharge flow channel 109, it is possible to reduce the pressure when the fluid is supplied. However, since the flow channel structure illustrated in part (d) of FIG. 15 can be produced by processing from one surface of the flow surface side, the flow channel structure illustrated in part (d) of FIG. 15 is superior to the flow channel structure illustrated in part (e) of FIG. 15 in terms of ease of production.

### Method for Producing Lipid Particle Encapsulating Drug

Hereinafter, a method for producing a lipid particle encapsulating a drug using the flow channel structure of the embodiment will be described.

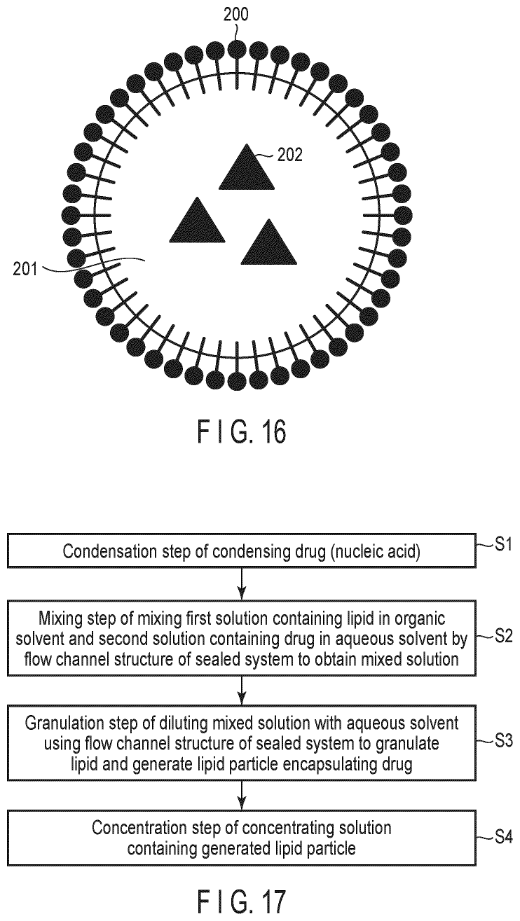

First, a lipid particle produced by the present method will be described. As shown in FIG. 16, a lipid particle 200 includes a lipid membrane formed by arranging lipid molecules, and has a substantially hollow spherical shape. A drug 202 is encapsulated in a lumen 201 of the lipid particle 200. The lipid particle 200 may be used, for example, to deliver the drug 202 into cells.

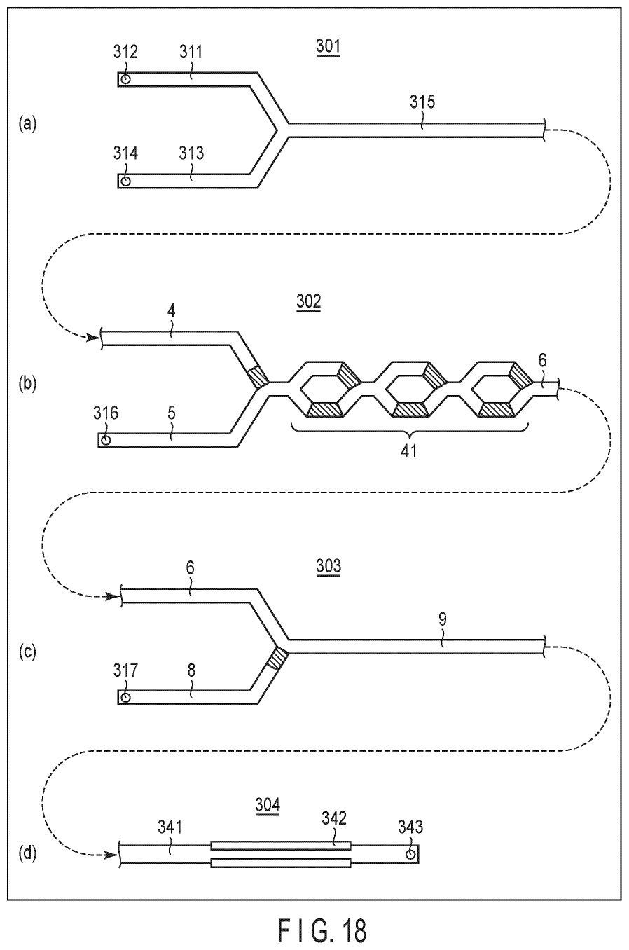

The production method includes, for example, the following steps as illustrated in FIG. 17:
condensing a drug (in the case of a nucleic acid) (condensation step S1);
mixing a first solution containing a lipid of a material of a lipid particle in an organic solvent and a second solution containing a drug in an aqueous solvent using a flow channel structure of a sealed system to obtain a mixed solution (mixing step S2);
decreasing a concentration of the organic solvent by mixing the mixed solution and an aqueous solvent using the flow channel structure of the sealed system to granulate the lipid and generate the lipid particle encapsulating the drug (granulation step S3); and
concentrating a solution containing the generated lipid particle (concentration step S4).

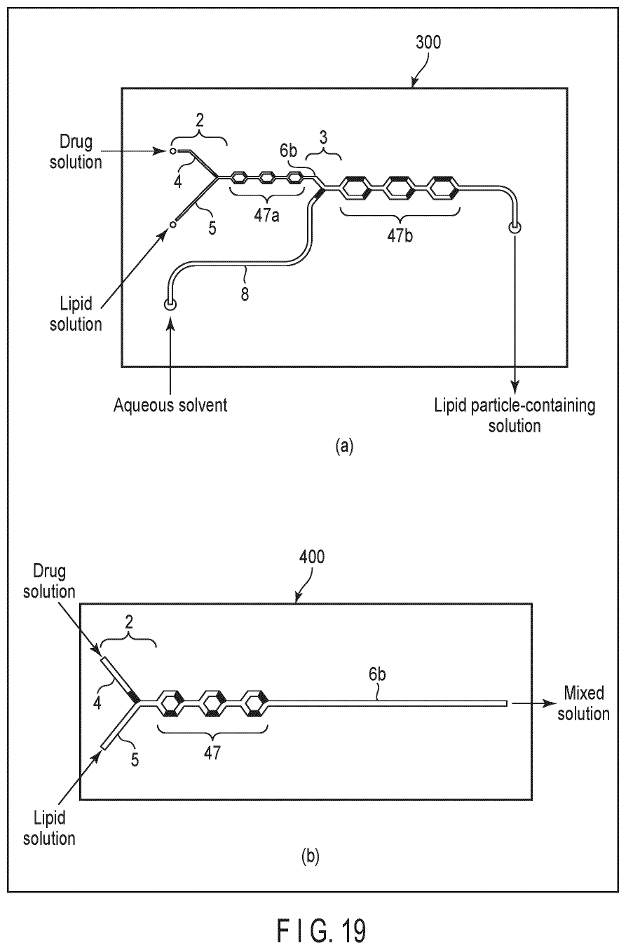

The present production method can be performed using, for example, a flow channel structure illustrated in FIG. 18. Part (a) of FIG. 18 illustrates an condensation flow channel 301 of the flow channel structure in which the condensation step S1 is performed, part (b) of FIG. 18 illustrates a mixing flow channel 302 in which the mixing step S2 is performed, part (c) of FIG. 18 illustrates a granulation flow channel 303 in which the granulation step S3 is performed, and part (d) of FIG. 18 illustrates a concentration flow channel 304 in which the concentration step S4 is performed.

Hereinafter, an example of procedures of the present production method will be described. First, the first solution and the second solution are prepared. The first solution contains a lipid in an organic solvent. The lipid is a lipid to be a material constituting the lipid particle 200. The second solution contains the drug 202 in an aqueous solvent.

### Condensation Step S1

The drug 202 is not limited, and is, for example, a nucleic acid. The drug 202 of the nucleic acid is, for example, a nucleic acid or the like containing DNA, RNA and/or other nucleotides, and may be, for example, mRNA of a specific gene, DNA encoding a gene, a vector or DNA containing a gene expression cassette including a gene and other sequences for expressing a gene such as a promoter, or the like. When the drug 202 is a nucleic acid, first, the condensation step S1 of condensing a nucleic acid (drug 202) may be performed.

The condensation of the nucleic acid is performed using, for example, a nucleic acid condensing peptide. The nucleic acid condensing peptide can further reduce the particle size of the lipid particle 200 by condensing the nucleic acid into a small size, and can encapsulate more nucleic acid in the lipid particle 200. As a result, the nucleic acid remaining outside the lipid particles 200 that may cause condensation of the lipid particles 200 may be reduced.

A preferred nucleic acid condensing peptide is, for example, a peptide containing a cationic amino acid in an amount of 45% or more of the whole peptide. A more preferred nucleic acid condensing peptide has RRRRRR (SEQ ID NO: 1, hereinafter, referred to as “first amino acid sequence”) at one end and a sequence RORQR (SEQ ID NO: 2, hereinafter, referred to as “second amino acid sequence”) at the other end. Between the first amino acid sequence and the second amino acid sequence, 0 or 1 or more intermediate sequences including RRRRRR or ROROR are contained. Two or more neutral amino acids are contained between two adjacent sequences among the first amino acid sequence, the second amino acid sequence, and the intermediate sequence. The neutral amino acid is, for example, G or Y. The other end may have RRRRRR (SEQ ID NO: 1, hereinafter, referred to as “first amino acid sequence”) in place of the second amino acid sequence.

The nucleic acid condensing peptide preferably has the following amino acid sequence:

| (SEQ ID NO: 3) | |
|---|---|
| | RQRQRYYRQRQRGGRRRRRR, |
| (SEQ ID NO: 4) | |
| | RQRQRGGRRRRRR, |
| | and |
| (SEQ ID NO: 5) | |
| | RRRRRRYYRQRQRGGRRRRRR. |

A nucleic acid condensing peptide having the following amino acid sequence GNQSSNFGPMKGGNFGGRSSGPYGGGGQYFAKPRNOGGY (SEQ ID NO: 6, hereinafter, referred to as “M9”) can also be used in combination with any of the nucleic acid condensing peptides described above. This peptide can further condense a nucleic acid condensate condensed with the nucleic acid condensing peptide.

As illustrated in part (a) of FIG. 13, the condensation flow channel 301 for performing the condensation step S1 is, for example, a Y-shaped flow channel. For example, an condensing agent inlet 312 is provided at an upstream end of one diverged flow channel 311 of the Y-shaped flow channel, and an condensing agent containing a nucleic acid condensing peptide flows from the condensing agent inlet 312. A drug inlet 314 is provided at an upstream end of the other flow channel 313, and a solution containing a nucleic acid (drug 202) in an aqueous solvent flows from the drug inlet 314. The aqueous solvent is, for example, water, saline such as physiological saline, a glycine aqueous solution, a buffer solution, or the like. As a result, a condensing agent and the solution containing the drug 202 are mixed in a flow channel 315 where the flow channel 311 and the flow channel 313 merge. By mixing, the second solution containing the condensed drug 202 is obtained.

The condensation step S1 is not necessarily performed using a flow channel, and in this step, the condensing agent and the solution containing a nucleic acid (drug 202) in an aqueous solvent may be mixed and stirred.

From the viewpoint of achieving the above effect, it is preferable to perform the condensation step S1 when the drug 202 is a nucleic acid. However, for example, when the drug 202 is not a nucleic acid or when the drug 202 is a nucleic acid but does not need to be condensed, it is not necessary to perform the condensation step S1.

### Mixing Step S2

Next, the first solution and the second solution are mixed.

The second solution may be prepared as described above when the drug 202 is a nucleic acid. Alternatively, in the case of using the drug 202 that is a nucleic acid not to be condensed or is not a nucleic acid, the second solution can be prepared by mixing the drug 202 with any of aqueous solvents selected according to the type of the drug. The drug 202 that is not a nucleic acid includes, for example, a protein, a peptide, an amino acid, another organic compound or inorganic compound, or the like as an active ingredient. The drug 202 may be, for example, a therapeutic agent or diagnostic agent for a disease. However, the drug 202 is not limited thereto, and may be any substance as long as it can be encapsulated in the lipid particle 200.

The drug 202 may further contain, for example, a reagent such as a pH adjusting agent, an osmotic pressure adjusting agent, and/or a drug activating agent, as necessary. The pH adjusting agent is, for example, an organic acid such as citric acid and a salt thereof. The osmotic pressure adjusting agent is a sugar, an amino acid, or the like. The drug activating agent is, for example, a reagent that assists the activity of the active ingredient. These agents may be added after the condensation step S1 when the condensation step S1 is performed.

The drug 202 may be a single substance or may include a plurality of substances. The concentration of the drug 202 in the second solution is preferably, for example, 0.01% to 1.0% (weight).

The first solution may be produced by mixing a lipid and an organic solvent. The lipid may be, for example, a lipid of a main component of a biological membrane. The lipid may be artificially synthesized. The lipid may include, for example, a phospholipid or a sphingolipid, for example, a base lipid such as diacylphosphatidylcholine, diacylphosphatidylethanolamine, ceramide, sphingomyelin, dihydrosphingomyelin, kephalin, cerebroside, or a combination thereof.

For example, as the base lipid,

it is preferable to use

1,2-dioleoyl-sn-glycero-3-phosphoethanolamine (DOPE),

1,2-stearoyl-sn-glycero-3-phosphoethanolamine (DSPE),

1,2-dipalmitoyl-sn-glycero-3-phosphatidylcholine (DPPC),

1-palmitoyl-2-oleoyl-sn-glycero-3-phosphatidylcholine (POPC),

1,2-di-O-octadecyl-3-trimethylammoniumpropane (DOTMA),

1,2-dioleoyl-3-dimethylammoniumpropane (DODAP),

1,2-dimyristoyl-3-dimethylammoniumpropane (14:0 DAP),

1,2-dipalmitoyl-3-dimethylammoniumpropane (16:0 DAP),

1,2-distearoyl-3-dimethylammoniumpropane (18:0 DAP),

N-(4-carboxybenzyl)-N, N-dimethyl-2,3-bis (oleoyloxy) propane (DOBAQ),

1,2-dioleoyl-3-trimethylammoniumpropane (DOTAP),

1,2-dioleoyl-sn-glycero-3-phosphochlorin (DOPC),

1,2-dilinoleoyl-sn-glycero-3-phosphochlorin (DLPC),

1,2-dioleoyl-sn-glycero-3-phospho-L-serine (DOPS),

cholesterol,

a combination of any of these, or the like. In particular, it is preferable to use DOTAP and/or DOPE.

The lipid preferably further includes a first lipid compound and/or a second lipid compound that are biodegradable lipids. The first lipid compound can be represented by formula:

$$Q - C H {R}_{2} ,$$

wherein
“Q” is a nitrogen-containing aliphatic group containing two or more tertiary nitrogens and no oxygen
“R” are each independently a C₁₂ to C₂₄ aliphatic group, and
at least one R includes, in its main chain or side chain, a linking group LR selected from the group consisting of —C(═O)—O—, —O—C(═O)—, —O—C(═O)—O—, —S—C(═O)—, —C(═O)—S—, —C(═O)—NH—, and —NHC(═O)—.

The first lipid compound is, for example, a lipid having a structure represented by the following formula.

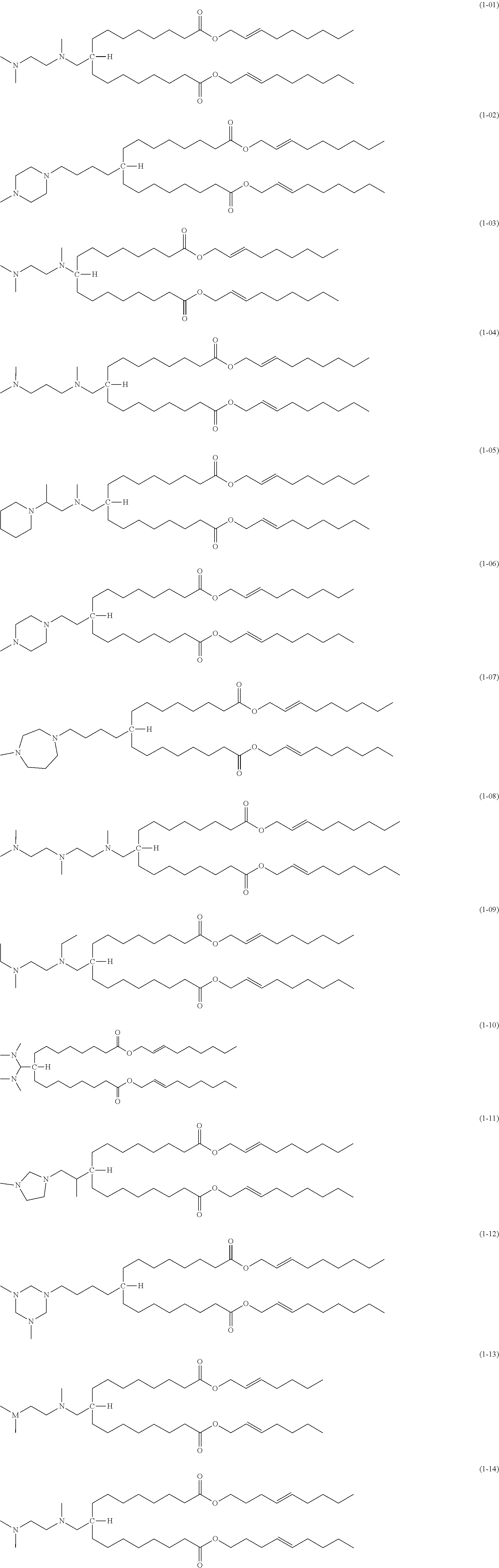

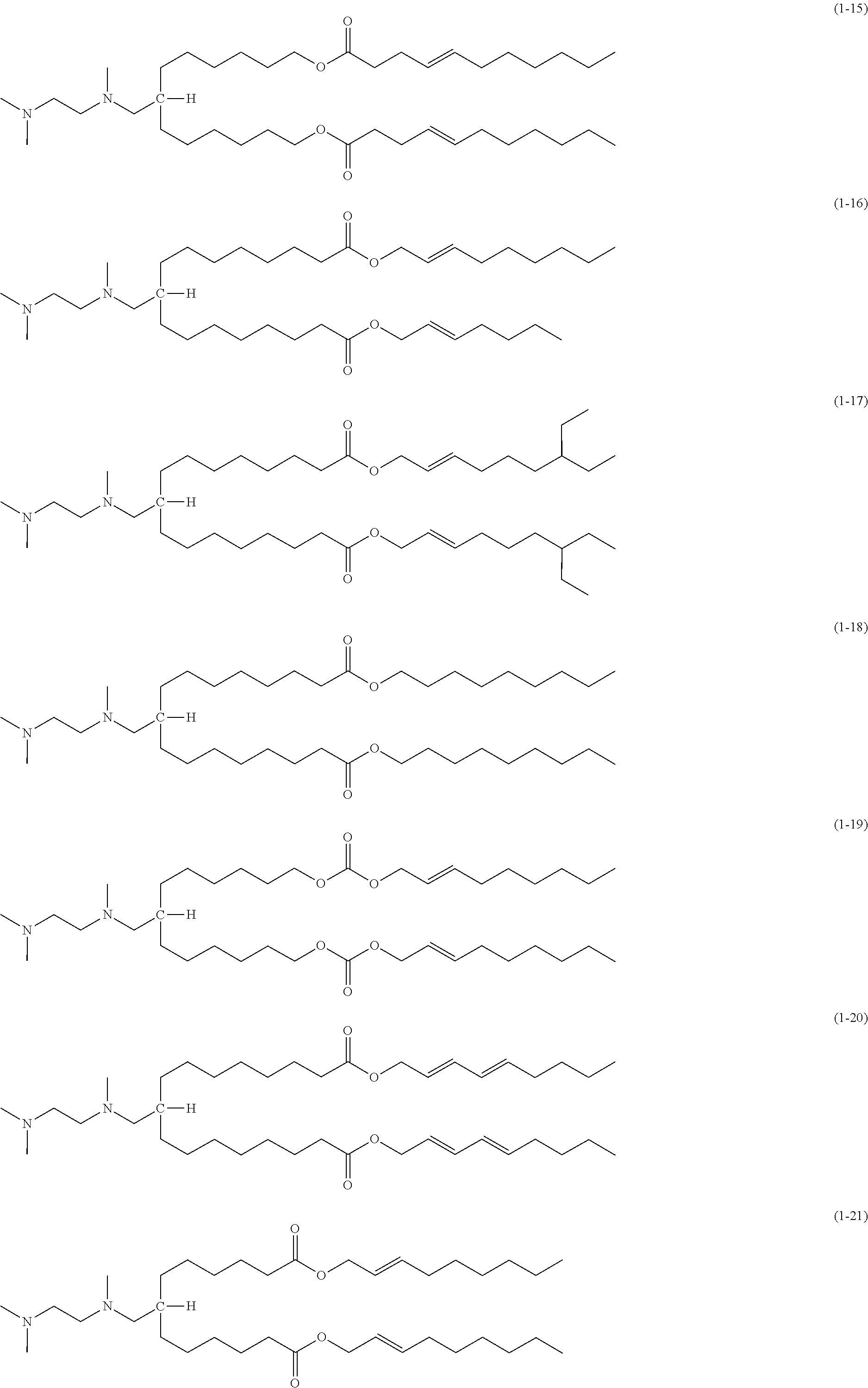

In particular, it is preferable to use a lipid compound of Formula (1-01) and/or a lipid compound of Formula (1-02).

The second lipid compound can be represented by formula:

$$P - {[ X - W - Y - {W}^{′} - Z ]}_{2} ,$$

wherein
“P” is alkyleneoxy containing one or more ether bonds in the main chain,
“X” are each independently a divalent linking group containing a tertiary amine structure,
“W” are each independently C₁ to C₆ alkylene,
“Y” are each independently a divalent linking group selected from the group consisting of a single bond, an ether bond, a carboxylic acid ester bond, a thiocarboxylic acid ester bond, a thioester bond, an amide bond, a carbamate bond, and a urea bond,
“W′” are each independently a single bond or a C₁ to C₆ alkylene, and
“Z” are each independently a fat-soluble vitamin residue, a sterol residue, or a C₁₂ to C₂₂ aliphatic hydrocarbon group.

The second lipid compound is, for example, a lipid having a structure represented by the following formula. In particular, it is preferable to use a compound of Formula (2-01).

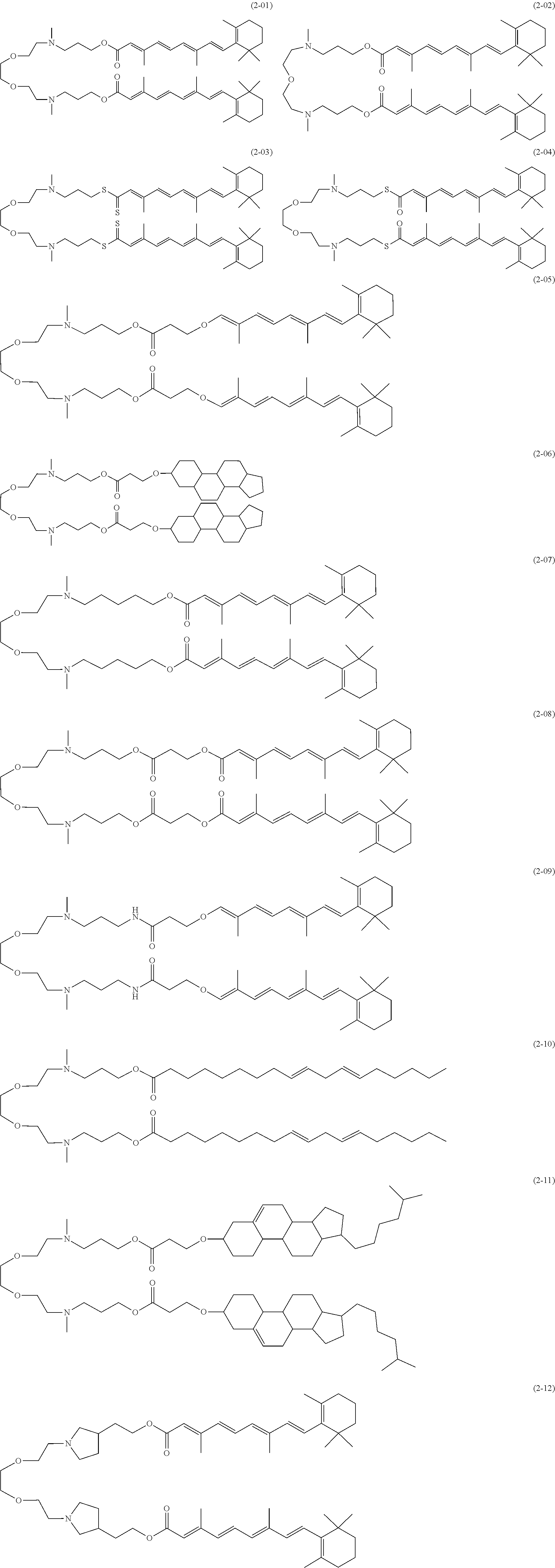

In the case of containing the first lipid compound and the second lipid compound, it is possible to increase the encapsulation amount of the drug 202 in the lipid particle 200 and to increase the introduction efficiency of the drug 202 into cells. Cell death of the introduced cells can also be reduced.

The base lipid is preferably contained in an amount of 30% to about 80% (molar ratio) with respect to all of the lipid materials. Alternatively, nearly 100% of the lipid material may be formed from the base lipid. The first and second lipid compounds are preferably contained in an amount of about 20% to about 70% (molar ratio) with respect to all of the lipid materials.

It is also preferable that the lipid includes a lipid preventing condensation of the lipid particles 200. For example, the lipid preventing condensation preferably further includes a PEG-modified lipid, for example, polyethylene glycol (PEG) dimyristoyl glycerol (DMG-PEG), a polyamide oligomer (U.S. Pat. No. 6,320,017 B) derived from an omega-amino (oligoethylene glycol) alkanoic acid monomer, monosialoganglioside, or the like. Such a lipid is preferably contained in an amount of about 1% to about 10% (molar ratio) with respect to all of the lipid materials of the lipid particle 200.

The lipid may further include lipids such as a lipid having relatively low toxicity for adjusting toxicity; a lipid having a functional group that binds a ligand to the lipid particle 200; and a lipid for suppressing leakage of an encapsulated product such as sterol, for example, cholesterol. In particular, it is preferable to contain cholesterol.

For example, the lipid particle 200 preferably contains the compound of Formula (1-01) or Formula (1-02) and/or the compound of Formula (2-01), DOPE and/or DOTAP, cholesterol, and DMG-PEG.

The type and constitution of the lipid are appropriately selected in consideration of an acid dissociation constant (pKa) of the intended lipid particle 200 or the size of the lipid particle 200, the type of an encapsulated product, stability in a cell to be introduced, and the like. For example, in order to obtain a desired constitution of a lipid constituting the lipid particle 200, the constitution of the lipid to be contained in the first solution may be set to the same ratio.

The organic solvent of the first solution is, for example, ethanol, methanol, isopropyl alcohol, ether, chloroform, benzene, acetone, or the like. The concentration of the lipid in the organic solvent is preferably, for example, 0.1% to 0.5% (weight).

The first solution and the second solution are mixed in the mixing flow channel 302 as illustrated in part (b) of FIG. 18. As the mixing flow channel 302, the same structure as the flow channel structure 40 described in the fourth embodiment has been shown, but the mixing flow channel 302 is not limited thereto, and may have, for example, the same structure as the flow channel structure 1 of the first embodiment, the flow channel structure 30 of the third embodiment, the flow channel structure 50 of the fifth embodiment, or the like.

When the condensation step S1 is performed, a downstream end of the flow channel 315 of the condensation flow channel 301 is connected to the upstream end of the first flow channel 4 of the flow channel structure 40, and the second solution is supplied to the first flow channel 4. When the condensation step S1 is not performed, a second solution inlet (not illustrated) is provided at the upstream end of the first flow channel 4, and the second solution is supplied from the second solution inlet. The second flow channel 5 includes, for example, a first fluid inlet 316 at the upstream end of the second flow channel 5, and the first solution is supplied from the first fluid inlet 316. As a result, as described in the fourth embodiment, in the first swirl structure 2 and a mixing unit 41, the first solution and the second solution are mixed and stirred to obtain a mixed solution. For example, when the condensation step S1 is not performed, the first solution may flow through the first flow channel 4, and the second solution may flow through the second flow channel 5.

### Granulation Step S3

Next, the concentration of the organic solvent of the mixed solution is decreased in the granulation step S3. For example, it is preferable to relatively decrease the organic solvent concentration by adding a large amount of the aqueous solution to the mixed solution. For example, an aqueous solution is added in an amount three times the amount of the mixed solution to the mixed solution. As the aqueous solution, the same aqueous solvent as that used for the first solution can be used. By decreasing the organic solvent concentration, the lipid can be granulated to generate the lipid particle 200 encapsulating the drug 202. As a result, a lipid particle-containing solution containing the lipid particle 200 is obtained.

As illustrated in part (c) of FIG. 18, the granulation flow channel 303 in which the granulation step S3 is performed is the same as the second swirl structure described in the first embodiment. The granulation flow channel 303 is not limited thereto, and may have, for example, the same structure as the flow channel structures of the second to fifth embodiments. The upstream end of the fourth flow channel 8 includes, for example, an aqueous solution inlet 317, and an aqueous solution is supplied from the aqueous solution inlet 317. As a result, the lipid is granulated to form the lipid particle 200 encapsulating the drug 202, thereby obtaining a lipid particle solution containing the lipid particle 200. As described above, the lipid particle 200 can be produced.

### Concentration Step S4

The method for producing a lipid particle of the embodiment may further include concentrating the lipid particle solution as necessary (concentration step S4). The concentration is performed, for example, by removing a part of the solvent and/or excess lipid and the drug 202 from the lipid particle solution. The concentration can be performed, for example, by ultrafiltration. For ultrafiltration, for example, an ultrafiltration filter having a pore diameter of 2 nm to 100 nm is preferably used. For example, Amicon (registered trademark) Ultra-15 (Merck) or the like can be used as the filter. By performing the concentration step S4, a lipid particle solution having high purity and concentration can be obtained. The concentration of the lipid particle 200 in the lipid particle solution after concentration is preferably about 1×1013 particles/mL to 5×1013 particles/mL. However, the concentration step S4 is not necessarily performed.

As illustrated in part (d) of FIG. 18, the concentration flow channel 304 in which the concentration step S4 is performed includes a flow channel 341 and a filter 342 provided on a wall surface of the flow channel 341. An upstream end of the flow channel 341 is connected to, for example, the end of the fifth flow channel 9 that is a downstream end of the granulation flow channel 303.

The filter 342 is provided instead of, for example, a partial wall surface of the flow channel 341. Any of the ultrafiltration filters described above can be used as the filter 342.

By making the lipid particle solution to pass through the flow channel 341, the remaining material, excess solvent, and the like pass through the filter 342 and are discharged to the outside of the flow channel 341. The lipid particles 200 remain in the flow channel 341 and flow downstream, so that the lipid particle solution is concentrated. A downstream end of the flow channel 341 may include a discharge port 343 configured to recover the lipid particle solution after concentration, or may be connected to a tank configured to recover the lipid particle solution.

The concentration step S4 is not necessarily performed using a flow channel, and for example, the lipid particle solution recovered in a container may be filtered by a filter.

In the method for producing a lipid particle of the embodiment, a treatment for improving the quality of the lipid particle 200 may be further performed as necessary. The improvement in quality can be, for example, prevention of leakage of the drug 202 from the lipid particle 200, improvement in the encapsulation amount of the drug 202 in the lipid particle 200, improvement in the ratio (encapsulation rate) of the lipid particle 200 encapsulating the drug 202, reduction and prevention of condensation of the lipid particles 200, and/or reduction in variation in the size of the lipid particles. For example, a treatment for cooling the lipid particle solution may be performed. Such a treatment may also be performed using a flow channel, and for example, the flow channel structure 50 including a flow channel having a long residence time described in the fifth embodiment may be used.

Each of the above-described flow channels is, for example, a micro flow channel. The flow of the fluid in the flow channel, the injection of the fluid into the flow channel, the extraction of the fluid from a tank, and/or the accommodation of the lipid particle solution in a container can be performed, for example, by a pump or an extrusion mechanism configured and controlled to automatically perform these operations.

The method for producing a lipid particle of the embodiment does not necessarily need to perform the condensation step S1 and the concentration step S4 as described above, and may include at least the mixing step S2 and the granulation step S3.

According to the method for producing a lipid particle of the embodiment, since the mixing step S2 is performed using the flow channel structure of the embodiment, the first solution and the second solution can be sufficiently mixed and stirred, and the lipid particles 200 having a more uniform particle size can be produced. When the particle size of the lipid particles 200 is more uniform, for example, effects such as improvement of the encapsulation amount of the drug 202, reduction of the average particle size of the lipid particles 200, and improvement of the proportion of the lipid particle encapsulating the drug 202 are expected. That is, it is possible to produce a lipid particle of higher quality by the method for producing a lipid particle of the embodiment.

As a further embodiment, a lipid particle may be produced using a flow channel structure further including the swirl structures (total number is m) with reference to the flow channel structure 1 of the first embodiment, that is, the flow channel structure according to the second embodiment.

A production method using the flow channel structure according to the second embodiment includes, for example, the following steps:
supplying a lipid solution or a plurality of kinds of drug solutions containing various drugs to each of the first flow channel, the second flow channel, the fourth flow channel, and each flow channel from a sixth flow channel to a (2m+2)-th flow channel of the flow channels A to obtain a mixed solution of the lipid solution and the plurality of kinds of drug solutions; and
decreasing a concentration of the organic solvent of the mixed solution obtained by sufficiently mixing a lipid solution and a plurality of kinds of drug solutions to granulate the lipid and generate the lipid particle uniformly encapsulating various drugs.

As a further embodiment, by using the flow channel structure according to the second embodiment, it is possible to produce lipid particles uniformly containing various drugs and produce a high-quality lipid particle preparation.

Hereinafter, experiments performed using the flow channel structure and the method for producing a lipid particle of the embodiment will be described.

### Example 1

Experiment 1. Comparison between Dilution in Sealed System and Dilution in Open System

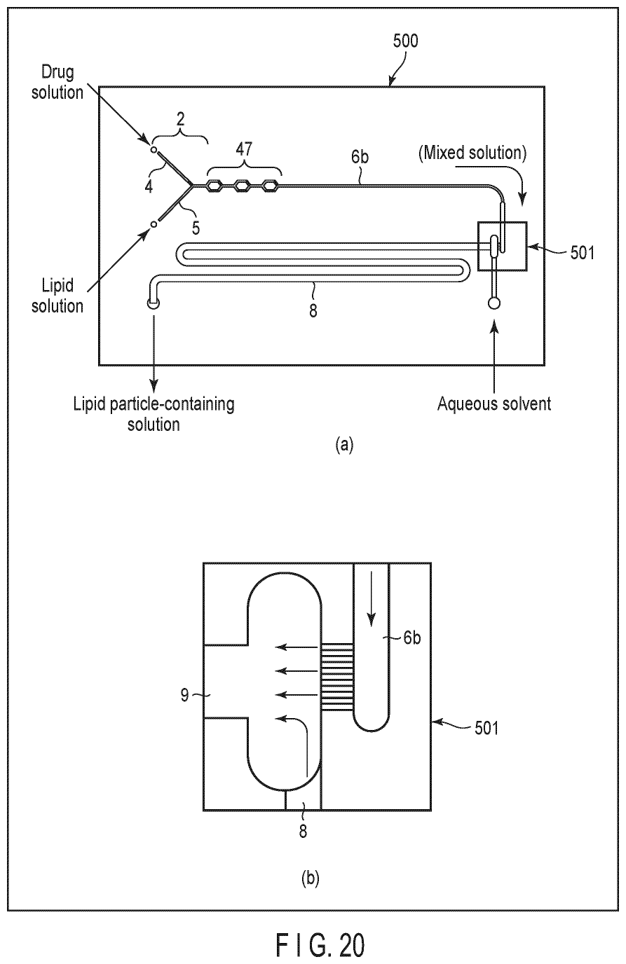

A flow channel structure 300 in which dilution can be performed in a sealed system as illustrated in part (a) of FIG. 19 was prepared. The flow channel structure 300 had the same configuration as that of the flow channel structure 49 according to the fourth embodiment. The dimensions of the flow channel structure 300 were made different between a combination of the first swirl structure 2 and the mixing unit group 47a and a combination of the second swirl structure 3 and the mixing unit group 47b in terms of the flow channel width and the size of the cross-sectional area. Specifically, the flow channel width of each of the first swirl structure 2 and the mixing unit group 47a was set to 0.3 mm and the flow channel width of each of the second swirl structure 3 and the mixing unit group 47b was set to 0.6 mm. However, the flow channel width of the third flow channel 6 is widened from 0.3 mm to 0.6 mm.

As a lipid solution, a solution obtained by dissolving a lipid composition of lipid constitution A (FFT20: DOPE: DOTAP: cholesterol: DNG-PEG2000=70:10:20:120:8 (molar ratio)) in ethanol was prepared.

An aqueous solution in which 0.1 mg/ml (5 kbp) double-stranded nucleic acid was dissolved in 9 times the amount of 10 mM HEPES (pH 7.3) was prepared as a drug solution.

As Example 1, the lipid solution containing a lipid and the drug solution containing a nucleic acid as a drug in an aqueous solvent were supplied to the first flow channel 4 and the second flow channel 5 of the flow channel structure 300, respectively, and the same aqueous solvent as the aqueous solvent of the drug solution was supplied to the fourth flow channel 8, thereby producing lipid particles.

The average particle size and the polydispersity index (pdi) of the produced lipid particles were measured in a particle size measuring mode of Zetasizer (registered trademark) Nano ZSP (Malvern Panalytical Ltd.). The higher the polydispersity index, the larger the variation in the particle size of the obtained lipid particles, and conversely, the smaller the polydispersity index, the more uniform the particle size of the lipid particles.

The nucleic acid encapsulation amount of the lipid particle was calculated by converting the relative ratio of fluorescence intensity obtained by measurement in the plasmid measurement mode of Quant-iTTM PicoGreen (registered trademark) ds DNA Assay Kit (Theermo Fisher Scientific).

### Comparative Example 1

As Comparative Example 1, a flow channel structure 400 illustrated in part (b) of FIG. 19 was prepared. Although the flow channel structure 400 has the same structure as the first swirl structure 2 and the mixing unit group 47a of the flow channel structure 300, the flow channel structure 400 does not have a structure corresponding to the second swirl structure 3 and the mixing unit group 47b of the flow channel structure 300, and dilution and the granulation step by the flow channel structure of the embodiment were not performed.

Instead, in Comparative Example 1, the mixed solution of the lipid solution and the drug solution discharged from the flow channel structure 400 was diluted by an operation of mixing with an aqueous solvent in the air (that is, an open system) to produce lipid particles. The average particle size, the polydispersity index, and the nucleic acid encapsulation amount of the lipid particles produced in Comparative Example 1 were measured by the same method as in Example 1.

### Example 2

As Example 2, a lipid solution having a lipid constitution different from that of Example 1 was supplied to the flow channel structure 300 of part (a) of FIG. 19 to produce lipid particles. As a lipid solution having a different lipid constitution, a solution obtained by dissolving a lipid composition of lipid constitution B (FFT10: FFT20: DOPE: DOTAP: cholesterol: DNG-PEG2000=35:70:21:9.4:88.5:9.4 (molar ratio)) in an ethanol was prepared and used. The constitution of the drug solution used for the lipid particle is the same as that in Example 1.

### Comparative Example 2

As Comparative Example 2, a lipid solution having a lipid constitution different from that of Comparative Example 1 was supplied to the flow channel structure 400 of part (b) of FIG. 19. As a lipid solution having a different lipid constitution, a solution obtained by dissolving a lipid composition of lipid constitution B (FFT10: FFT20: DOPE: DOTAP: cholesterol: DNG-PEG2000=35:70:21:9.4:88.5:9.4 (molar ratio)) in an ethanol was prepared and used. The constitution of the drug solution used for the production of lipid particles in Comparative Example 2 is the same as those in Example 1, Example 2, and Comparative Example 1. The lipid particles were prepared by the same method as in Comparative Example 1.

The average particle size, the polydispersity index, and the nucleic acid encapsulation amount of the lipid particles produced in Example 2 and Comparative Example 2 were measured by the same method as in Example 1.

### Results

Table 1 shows the experimental results of Example 1.

TABLE 1

| | | | Nucleic acid |
|---|---|---|---|
| | Particle | Polydispersity | encapsulation |
| | size (nm) | index (−) | amount (μg/mL) |
| Sealed system Lipid | 90.85 | 0.04 | 356.0 |
| constitution A | | | |
| (Example 1) | | | |
| Open system Lipid | 91.28 | 0.11 | 230.4 |
| constitution A | | | |
| (Comparative Example 1) | | | |
| Sealed system Lipid | 64.50 | 0.14 | 437.1 |
| constitution B | | | |
| (Example 2) | | | |
| Open system Lipid | 88.80 | 0.15 | 436.5 |
| constitution B | | | |
| (Comparative Example 2) | | | |

Comparing the results between Example 1 and Comparative Example 1, it is found that Example 1 has a lower polydispersity index than Comparative Example 1, and the particle size of the lipid particles produced in Example 1 is more uniform. It is found that in Example 1, the average particle size of the produced lipid particles was slightly smaller than that in Comparative Example 1, but the nucleic acid encapsulation amount was significantly improved.

Similarly, comparing the results between Example 2 and Comparative Example 2, Example 2 has a lower polydispersity index than Comparative Example 2, and the particle size of the lipid particles produced in Example 2 is more uniform. It is found that in Example 2, the average particle size of the produced lipid particles was smaller than that in Comparative Example 2, but the nucleic acid encapsulation amount was slightly increased.

Therefore, it was shown that dilution and granulation using the flow channel structure of the sealed system (Examples 1 and 2) can produce lipid particles with better quality than the production method in which preparation of the mixed solution using the flow channel structure and dilution in the open system are combined (Comparative Examples 1 and 2).

In Example 1 and Example 2, lipid solutions having different constitutions were supplied, but it was shown that both of the lipid solutions can be applied to solutions having different lipid constitutions since lipid particles having good quality could be produced.

Experiment 2. Comparison between Dilution by Swirl Structure and Dilution by Laminar Flow Structure

### Comparative Example 3

A flow channel structure 500 illustrated in part (a) of FIG. 20 was prepared. Although the flow channel structure 500 has the same structure as the first swirl structure 2 and the mixing unit group 47 of the flow channel structures 300 and 400, the flow channel structure 500 does not has a structure corresponding to the second swirl structure 3 and the mixing unit group 47b of the flow channel structure 300, but instead, includes a laminar flow structure 501, and a lipid solution was diluted by being mixed with an aqueous solvent in the laminar flow structure 501.

The laminar flow structure 501 refers to a flow channel merging portion configured such that swirling does not occur when a solution is mixed. As illustrated in part (b) of FIG. 20, the laminar flow structure 501 is configured such that the flow of the fluid does not collide with the wall surface. The flow channel structure 500 is different from the flow channel structure 400 in that the flow channel structure includes a flow channel in which dilution and granulation are performed.

As Comparative Example 3, a lipid solution, a drug solution, and an aqueous solvent were supplied to the flow channel structure 500 to produce a lipid particle. The constitutions of the lipid solution, the drug solution, the aqueous solvent, the experimental conditions such as a supply flow rate, and further, the measurement items and the measurement conditions are also the same as those in Example 1.

### Results

Table 2 shows the experimental results of Example 2.

TABLE 2

| | | | Nucleic acid |
|---|---|---|---|
| | Particle | Polydispersity | encapsulation |
| | size (nm) | index (−) | amount (μg/mL) |
| Sealed system | 90.85 | 0.04 | 356.0 |
| Swirl structure | | | |
| (Example 1) | | | |
| Sealed system | 96.78 | 0.08 | 298.8 |
| Laminar flow structure | | | |
| (Comparative Example 3) | | | |

Comparing the results between Example 1 and Comparative Example 3, it is found that Example 1 has a lower polydispersity index than Comparative Example 3, and the particle size of the lipid particles produced in Example 1 is more uniform. It is found that in Example 1, the average particle size of the produced lipid particles was smaller than that in Comparative Example 3, but the nucleic acid encapsulation amount was significantly improved.

Therefore, it was shown that dilution and granulation using the flow channel structure including a swirl structure (Example 1) can produce lipid particles with better quality than dilution and granulation using the flow channel structure including a laminar flow structure (Comparative Examples 1 and 2).

Experiment 3. Comparison between Shape Patterns of Swirl Structures

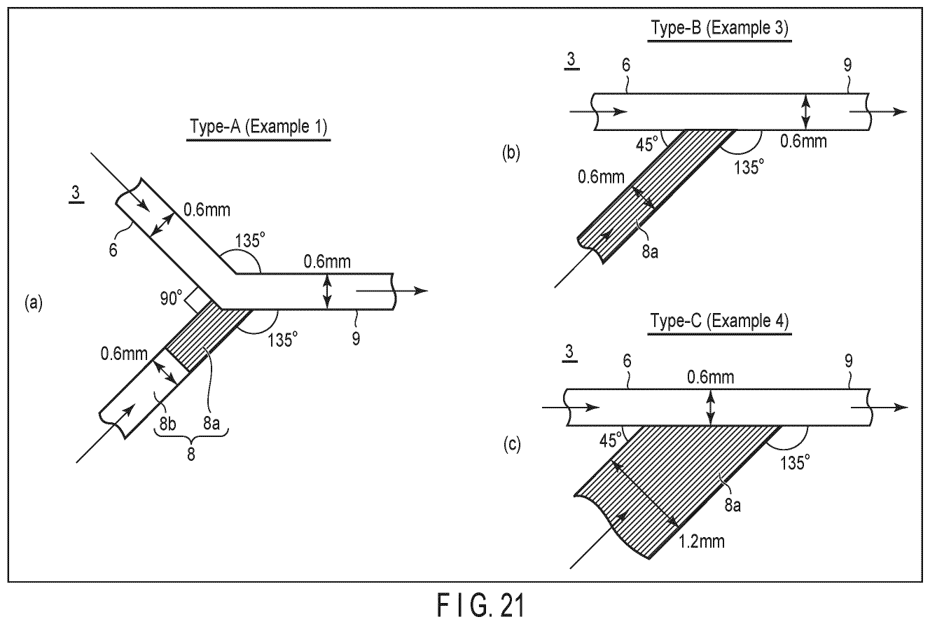

In order to verify whether the shape pattern of the swirl structure affects the dilution effect, two flow channel structures each including the second swirl structure 3 having a shape pattern different from the shape pattern of the second swirl structure 3 of Example 1 illustrated in part (a) of FIG. 21 (hereinafter, referred to as “Type-A”) were further prepared. Specifically, a flow channel structure including the second swirl structure 3 having a shape pattern illustrated in part (b) of FIG. 21 (hereinafter, referred to as “Type-B”) and a flow channel structure having a shape pattern illustrated in part (c) of FIG. 21 (hereinafter, referred to as “Type-C”) were prepared. Both the flow channel structures have the same configuration as in Example 1, except for the shape pattern of the second swirl structure.

### Example 3

As Example 3, a lipid particle was produced under the same conditions as in Example 1, except that the flow channel structure including the second swirl structure of Type-B was used.

Type-B has a shape in which the fourth flow channel 8 is connected to the third flow channel 6 and the fifth flow channel 9 extending to ride on the same straight line (that is, an angle formed by the third flow channel 6 and the fifth flow channel 9 is 180º). The flow channel width of each of the third flow channel 6, the fourth flow channel 8, and the fifth flow channel 9 of Type-B is 0.6 mm, which is the same as the structure of Type-A.

### Example 4

As Example 4, a lipid particle was produced under the same conditions as in Example 1, except that the flow channel structure including the second swirl structure of Type-C was used.

Similarly to Type-B, Type-C has a shape in which the fourth flow channel 8 is connected to the third flow channel 6 and the fifth flow channel 9 extending to the same straight line (that is, an angle formed by the third flow channel 6 and the fifth flow channel 9 is 180°). However, Type-C had an asymmetric swirl structure in which the flow channel width of each of the third flow channel 6 and the fifth flow channel 9 is 0.6 mm and the flow channel width of the fourth flow channel 8 is 1.2 mm. In the respect, the shape of Type-C is different from the shapes of Type-A and Type-B.

### Results

Table 3 shows the experimental results of Example 3.

TABLE 3

| | | | Nucleic acid |
|---|---|---|---|
| | Particle | Polydispersity | encapsulation |
| | size (nm) | index (−) | amount (μg/mL) |
| Type-A | 90.85 | 0.04 | 356.0 |
| (Example 1) | | | |
| Type-B | 86.30 | 0.06 | 380.6 |
| (Example 3) | | | |
| Type-C | 84.93 | 0.05 | 373.0 |
| (Example 4) | | | |

No large difference was observed in the average particle size, the polydispersity index, the nucleic acid encapsulation amount among the lipid particles produced in Example 1, Example 3, and Example 4. Therefore, it was shown that dilution can be effectively performed with any of the shape patterns Type-A, Type-B, and Type-C of the swirl structure.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A flow channel structure comprising:
a first flow channel;
a second flow channel;
a third flow channel;
a first merging portion that connects one end of the first flow channel, one end of the second flow channel, and one end of the third flow channel to one another;
a fourth flow channel;
a fifth flow channel;
a second merging portion that connects another end of the third flow channel, one end of the fourth flow channel, and one end of the fifth flow channel to one another; and
a second shallow portion that is located at the one end of the fourth flow channel and is shallower than a depth of the second merging portion.

2. The flow channel structure according to claim 1, wherein a depth of the first shallow portion is less than ½ of the depth of the first merging portion and/or a depth of the second shallow portion is less than ½ of the depth of the second merging portion.

3. The flow channel structure according to claim 1, wherein a cross-sectional area of the fourth flow channel is larger than a cross-sectional area of any of the first flow channel, the second flow channel, and the third flow channel.

4. The flow channel structure according to claim 1, wherein the third flow channel further includes a mixing unit,
the mixing unit includes a first diverging and merging flow channel and a second diverging and merging flow channel that are two flow channels diverging from the third flow channel and merging into the third flow channel again between the one end and said another end of the third flow channel, and
the first diverging and merging flow channel and the second diverging and merging flow channel each include:
a diverging portion that diverges from the third flow channel;
a merging portion that merges into the third flow channel;
an intermediate portion that connects the diverging portion and the merging portion and bends a connection part;
a third shallow portion that is located at an end of the first diverging and merging flow channel on the merging portion side and is shallower than a depth of the third flow channel; and
a fourth shallow portion that is located at the intermediate portion of the second diverging and merging flow channel and is shallower than depths of the diverging portion and the merging portion of the second diverging and merging flow channel.

5. The flow channel structure according to claim 4, wherein the merging portion of each of the first diverging and merging flow channel and the second diverging and merging flow channel into the third flow channel is connected to the third flow channel at an angle symmetrical to each other with respect to a long axis of the third flow channel.

6. The flow channel structure according to claim 4, wherein the third flow channel includes a plurality of the mixing units, and the mixing units are connected to each other in series and/or in parallel.

7. The flow channel structure according to claim 1, wherein the third flow channel further includes a flow channel that takes longer time for a solution to pass through than the first flow channel, the second flow channel, the fourth flow channel, and the fifth flow channel.

8. The flow channel structure according to claim 7, wherein the flow channel that takes longer time for a solution to pass through has a longer flow channel length and/or a wider flow channel width than the first flow channel, the second flow channel, the fourth flow channel, and the fifth flow channel.

9. The flow channel structure according to claim 1, further comprising
a trap structure configured to trap a foreign substance in the first flow channel on a side closer to another end than the first shallow portion,
wherein a depth of the trap structure is shallower than a depth of the first flow channel on a side closer to said another end than the first shallow portion.

10. The flow channel structure according to claim 9, further comprising the trap structure in the fourth flow channel on a side closer to another end than the second shallow portion and/or the second flow channel on a side closer to said another end than the first merging portion.

11. The flow channel structure according to claim 1, further comprising:
a flow channels A (total number is m, hereinafter, m is any integer of 1 or more) having a shallow portion on another end side of the fifth flow channel; and a flow channels B (total number is m) each of which is connected to each of the flow channels A,
wherein, when each of the flow channels A (total number is m) is represented as a (2n+4)-th flow channel (hereinafter, n is an integer of 1 to m), and each of the flow channels B (total number is m) is represented as a (2n+5)-th flow channel,
another end of a (2n+3)-th flow channel, one end of the (2n+4)-th flow channel, and one end of the (2n+5)-th flow channel are connected to one another in a (n+2)-th merging portion, and
the one end of the (2n+4)-th flow channel includes a (n+2)-th shallow portion that is shallower than a depth of the (n+2)-th merging portion.

12. A method for producing a lipid particle encapsulating a drug using the flow channel structure according to claim 1, the method comprising:
supplying a first solution containing a lipid of a material of the lipid particle in an organic solvent from one of the first flow channel and the second flow channel, supplying a second solution containing the drug in an aqueous solvent from the other one of the first flow channel and the second flow channel, and mixing the first solution and the second solution to obtain a mixed solution; and
decreasing a concentration of the organic solvent of the mixed solution by supplying an aqueous solvent from the fourth flow channel and diluting the mixed solution to granulate the lipid and generate the lipid particle encapsulating the drug.

13. The method according to claim 12, wherein the drug is a nucleic acid, and the method further comprises condensing the nucleic acid before mixing the first solution and the second solution.

14. The method according to claim 12, further comprising concentrating a lipid particle-including solution after the granulation.

15. A method for producing a lipid particle encapsulating a drug using the flow channel structure according to claim 11, the method comprising:
supplying a lipid solution containing a lipid of a material of the lipid particle in an organic solvent or a drug solution containing the drug in an aqueous solvent to each of the first flow channel, the second flow channel, the fourth flow channel, and each flow channel from a sixth flow channel to a (2m+2)-th flow channel of the flow channels A to obtain a mixed solution of the lipid solution and the drug solution; and
decreasing a concentration of the organic solvent of the mixed solution by supplying an aqueous solvent from the (2m+4)-th flow channel to granulate the lipid and generate the lipid particle encapsulating the drug.


SEQUENCE LISTING (6 sequences)

SEQ ID NO: 1 - protein, 6 aa, synthetic construct
  source 1..6: note = The first amino-acid sequence
RRRRRR

SEQ ID NO: 2 - protein, 5 aa, synthetic construct
  source 1..5: note = The second amino-acid sequence
RQRQR

SEQ ID NO: 3 - protein, 20 aa, synthetic construct
  source 1..20: note = nucleic acid condensed peptide
RQRQRYYRQRQRGGRRRRRR

SEQ ID NO: 4 - protein, 13 aa, synthetic construct
  source 1..13: note = nucleic acid condensed peptide
RQRQRGGRRRRRR

SEQ ID NO: 5 - protein, 21 aa, synthetic construct
  source 1..21: note = nucleic acid condensed peptide
RRRRRRYYRQRQRGGRRRRRR

SEQ ID NO: 6 - protein, 39 aa, Homo sapiens
  source 1..39: note = nucleic acid condensed peptide
GNQSSNFGPMKGGNFGGRSSGPYGGGGQYFAKPRNQGGY